(19) United States
(12) Patent Application Publication
(10) Pub. No.: US 2022/0401536 A1
(43) Pub. Date: Dec. 22, 2022

(54) USE OF DD1ALPHA (VISTA) MODULATORS IN CANCER TREATMENT: IMMUNOTHERAPY BASED ON THE DISRUPTION OF DD1ALPHA/PD-1 SIGNALING

(71) Applicant: Sam LEE, Bellevue, WA (US)

(72) Inventor: Sam LEE, Bellevue, WA (US)

(21) Appl. No.: 17/842,235

(22) Filed: Jun. 16, 2022

Related U.S. Application Data
(60) Provisional application No. 63/211,178, filed on Jun. 16, 2021.

(51) Int. Cl. A61K 39/00

(52) U.S. Cl. CPC A61K 39/0011

(57) ABSTRACT

Provided herein are compositions and methods for treating immune diseases or disorders, including cancer. In some embodiments, the compositions and methods are directed to the use of anti-DD1α antibodies to modulate the immune-regulatory activity of DD1α, thereby promoting anti-tumor immunity. Methods for identifying patients that are likely to respond to anti-DD1α therapy, and for treating such patients with anti-DD1α antibody compositions are also provided.

### CROSS-REFERENCE TO RELATED APPLICATIONS

This application claims the benefit of U.S. provisional application Ser. No. 63/211,178 filed Jun. 16, 2021, the disclosure of which is hereby incorporated in its entirety by reference herein.

### STATEMENT REGARDING FEDERALLY SPONSORED RESEARCH OR DEVELOPMENT

The invention was made with Government support under Contract Nos. CA203552 and CA210560 awarded by the National Institutes of Health. The Government has certain rights to the invention.

### SEQUENCE LISTING

The text file lees ST25 of size 23 KB created Jun. 16, 2022 filed herewith, is hereby incorporated by reference.

### TECHNICAL FIELD

In at least one aspect, the present invention relates to methods and compositions for treating immune-related diseases such as cancer, including identifying a subject having a cancer that would likely benefit from anti-DD1α therapy, and providing a composition containing antibodies against DD1α to the subject to antagonize intercellular interactions between members of the B7 family of immune-regulatory ligands, including DD1α and PD-1.

### BACKGROUND

Immune checkpoint regulators control the activation of immune cells to preserve the balance between immune tolerance and necessary immune responses. During cancer growth and progression, various immune cells infiltrate the tumor microenvironment. The immune surveillance mechanisms of these cells are often suppressed, leading to the loss of anti-tumor immune responses. Methods by which tumor cells downregulate anti-tumor immune responses include shifting the balance of activity from stimulatory immunoreceptors to inhibitory immunoreceptors. Blocking the activity of inhibitory immunoreceptors has shown promise as an effective addition to cancer treatment regimens for certain types of cancers. The response rate to inhibitory immunoreceptor blockade therapy, however, remains extremely low in many types of cancers. Accordingly, there is a need for new and more effective immune checkpoint regulatory therapeutics, based on a deeper understanding of the mechanisms utilized by immune checkpoint proteins.

### SUMMARY

In at least one aspect, the present invention provides a composition for treating immune-related diseases including cancer, comprising at least one isolated monoclonal antibody that modulates DD1α activity by binding to a DD1α polypeptide consisting of SEQ ID NO:1. In certain embodiments, the binding of the isolated monoclonal antibody to the DD1α polypeptide disrupts the physical association between the DD1α polypeptide on the surface of a first cell and a separate polypeptide on the surface of a second cell, or between the DD1α polypeptide on the surface of a first cell and a PD-1 polypeptide on the surface of a second cell. The composition additionally includes a pharmaceutically acceptable carrier or diluent. In another embodiment, the composition can include a pharmaceutically acceptable anti-cancer therapeutic.

In another aspect, the present invention provides a method for providing immunotherapy to a subject having cancer. The method comprises the steps of identifying a subject with a cancer that is likely to respond to the immunotherapy, and administering at least one isolated monoclonal antibody to the subject. In an embodiment, the likelihood of response to the immunotherapy is determined by obtaining a bodily fluid or tissue sample from a patient diagnosed with cancer, measuring the fraction of tumor cells expressing DD1α on the surface of the cell and comparing the fraction to a reference sample. If the fraction of tumor cells expressing DD1α on the surface of the cell exceeds a determined threshold, a composition containing the isolated monoclonal antibody is administered to the subject. In a further embodiment, the at least one isolated monoclonal antibody binds to a DD1α polypeptide consisting of SEQ ID NO:1. Additionally, the binding of the isolated monoclonal antibody to the DD1α polypeptide disrupts the physical association between the DD1α polypeptide on the surface of a first cell and a DD1α or PD-1 polypeptide on the surface of a second cell. In one embodiment, the composition administered to a patient identified as having a cancer likely to respond to anti-DD1α immunotherapy comprises the at least one isolated monoclonal antibody that disrupts the DD1α-DD1α or DD1α-PD-1 interactions, and a pharmaceutically acceptable carrier or diluent. In another embodiment, the composition further comprises a pharmaceutically acceptable anti-cancer therapeutic.

Previously, anti-PD-1 therapeutics have been used alone and in combination with an anti-cancer therapeutic to treat cancer. Such treatments, however, do not target the intercellular interaction between DD1α molecules or between DD1α and PD-1. In this way, cancer cells that overexpress DD1α, PD-1, and/or PD-L1 in patients being treated with anti-PD-1 therapeutics can still avoid immune surveillance mediated through the DD1α signaling pathway, unless the interaction of DD1α with DD1α and/or PD is also targeted. Accordingly, a new composition and method for treating cancer is therefore provided herein. The composition and method provide monoclonal antibodies that specifically inhibit the interaction of DD1α on the surface of a cancer cell with DD1α and/or PD-1 expressed on the surface of an immune cell (e.g. but not limited to, a T cell). Inhibiting this specific interaction reduces the ability of DD1α-expressing cancer cells to evade immune surveillance, thus improving patient prognoses.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIGS. 1A-1, 1A-2, 1A-3, 1A-4, 1A-5, 1A-6, 1A-7, 1A-8, 1A-9, 1B-1, 1B-2, 1B-3, and 1B-4. These figures show that DD1α is involved in mediating T cell tolerance. (A) PD-1 and PD-L1 are not involved in removal of apoptotic cells. Engulfment of apoptotic thymocytes by mouse bone marrow-derived macrophages (m-BMDMs) isolated from Wt, DD1α^{-/-}, PD-1^{-/-} and PD-L1^{-/-} mice was assessed by flow cytometry analysis. Thymocytes isolated from Wt mice were treated with 600 nM camptothecin (CPT) for 12 hours to induce apoptotic populations. The pHrodo-labeled mouse thymocytes (live or apoptotic) were incubated with Wt, DD1α^{-/-}, PD-L1^{-/-}, and PD-1^{-/-} m-BMDMs for 30 min. Phagocytosis was quantified by the percentage of macrophages containing positive pHrodo signal. Data are shown as mean±SD and representative of three independent experiments. (B) DD1α deficiency prevents in vivo induction of OVA peptide-specific tolerance of CD4⁺ T cells. Schematic diagram shows OVA peptide immunization and OVA peptide antigen-specific tolerance induction. OT-II Rag2^{-/-}, OT-II Rag2^{-/-}DD1α^{-/-} and OT-II Rag2^{-/-}PD-1^{-/-} mice were inoculated with OVA₃₂₃₋₃₃₉ (500 μg) or PBS on day 1 and day 4. On day 10, CD4⁺ T cells were isolated from spleen and lymph nodes of these mice and re-stimulated with OVA peptide antigen (0-10 μg/ml) for 72 hours. OVA specific T cell proliferation responses were chased by treatment with 10 μM 5-ethynyl-2′-deoxyuridine (EdU) and analyzed by Alexa Fluor 488 Click-iT Plus flow cytometry assay kits.

FIGS. 2A, 2B, 2C-1, 2C-2, 2D, 2E-1, 2E-2, 2E-3, 2E-4, 2F-1, 2F-2, 2F-3, 2F-4, 2F-5, 2F-6, 2F-7, 2F-8, 2F-9, 2G-1, and 2G-2. These figures show that the PD-1 receptor is identified as a novel DD1α binding partner. (A) Binding partners of DD1α identified by mass spectrometry following Ig pull-down assay. Membrane protein fractions from HCT116 cells were incubated with DD1α-Ig-bound beads and washed with binding buffer. A control binding reaction was performed in parallel using Ig only as bait protein. DD1α binding proteins were subjected to 4˜12 NuPAGE gel and were stained with Coomassie blue. The binding proteins were identified by mass spectrometric analysis. (B) Binding of DD1α to PD-1 by Ig pull-down. Control Ig (Ctrl-Ig), DD1α-Ig, PD-L1-Ig, and PD-1-Ig proteins were bound to Protein A/G Sepharose beads. For Ig pull-down assays, lysates from 293T cells transfected with DD1α-HA, PD-1-HA, or TIM3-HA (negative control) were incubated with the Protein A/G Sepharose-bound Ig proteins and the beadbound lysates were subjected to immunoblotting. (C) Co-immunoprecipitation of DD1α and PD-1. DD1α-Myc and PD-1-HA were transfected into 293T cells. DD1α-Myc proteins were immunoprecipitated with crosslinked anti-Myc agarose. The immunoprecipitates were resolved by SDS-PAGE and blotted against the indicated Abs. The reciprocal co-IP was performed using cross-linked anti-HA agarose. (D) In vitro binding between DD1α and PD-1. Proteins were expressed as Ig- or 6×-His fusion in 293T cells and bead-purified. Ctrl-Ig and PD-1-Ig fusion proteins were bound to Protein A/G Sepharose and incubated with 6×His-DD1α 33-194 at 4° C. O/N. After washing, resin-bound proteins were resolved on an SDS-PAGE and blotted against antiHis antibodies. (E) Immuno-EM analysis of the intracellular interaction between DD1α and PD-1. 293T cells were transiently co-transfected with expression constructs encoding Myc-fused DD1α and HA-fused PD-1, and prepared for double immunogold labeling with anti-Myc (10 nm gold) and anti-HA (15 nm gold) (top diagram). Arrowheads indicate the gold particles. Cells transfected with DD1α-Myc alone or PD-1-HA alone were used as controls. Scale bar, 100 nm. (F) Intercellular DD1α-PD-1 association and the disruption by recombinant PD-1 protein. The exogenous expression of DD1α or PD-1 on cells was validated by flow cytometry analysis (left). CFSE-labeled DD1α-expressing Jurkat cells were pre-incubated with Ig proteins (control Ig: 40 mg/ml; PD-1-Ig: 20 mg/ml and 40 mg/ml) for 30 min and mixed with Far Red-labeled control or PD-1-expressing 293A cells. After 1 hour incubation, DD1α-PD-1-mediated intercellular bindings were analyzed by counting the percentage of CFSE- and Far Red-positive populations (right). (G) Binding of blue latex beads coated with Ig, DD1α-Ig, or PD-1-Ig fusion proteins (the extracellular region of DD1α or PD-1 fused with the immunoglobulin G Fc segment) to 293T cells transfected with empty vector (EV), a mutant DD1α lacking the IgV domain (DD1α-ΔIgV), or Wt DD1α. Ig protein-coated beads were included as a control. After 30 min, unbound beads were washed. The binding was examined under an inverted microscope (top) and also determined from the optical density (O.D.) at 492 nm (bottom). Data are shown as mean±SD and are representative of three experiments.

FIGS. 3A-1, 3A-2, 3A-3, 3A-4, 3A-5, 3A-6, 3A-7, 3A-8, 3A-9, 3A-10, 3A-11, 3A-12, 3A-13, 3A-14, 3A-15, 3A-16, 3A-17, 3A-18, 3A-19, 3A-20, 3B-1, 3B-2, 3B-3, 3B-4, 3B-5, 3B-6, 3B-7, 3B-8, 3B-9, 3B-10, 3B-11, 3B-12, 3B-13, 3B-14, 3B-15, 3B-16, 3B-17, 3B-18, 3B-19, 3B-20, 3B-21, 3B-22, 3B-23, 3B-24, 3B-25, 3B-26, 3B-27, 3B-28, 3B-29, and 3B-30. These figures show that the bidirectional ligand/receptor interaction between DD1α and PD-1 regulates T cell proliferation. (A) DD1α binds less efficiently to PD-1-deficient T cells and vice versa: DD1α-deficient T cells bind less to PD-1. CD4+ and CD8+ T cells from Wt or DD1α−/− or PD-1−/− mice were stimulated by plate-bound 5 μg/ml CD3-specific Ab for 3 days. Stimulated T cells were assayed for bindings to Ig-fused DD1α, PD-1, and PD-L1-Ig proteins. Cells were incubated with DD1α-Ig, PD-1-Ig, or PD-L1 (unshaded in the histograms), or with control Ig, and the Ig-fused protein-bound cells were detected by Ab against Ig-PE. The PE labeled anti-IgG antibody specifically detects Ig-fused proteins. This analysis is performed in the flow cytometer. Gray filled in the histograms represents control-Ig bound cells, and unshaded in the histograms represents DD1α-Ig, PD-1-Ig and PD-L1-Igbound cells. Data are shown as mean±SD and representative of three independent experiments. (B) DD1α-mediated T cell suppression is reversed in PD-1-deficient T cells, and likewise PD-1-mediated T cell suppression is attenuated in DD1α-deficient T cells. CD4⁺ T cells or CD8+ T cells from Wt or PD-1-deficient or DD1α-deficient mice were stained with CFSE (1 μM) and stimulated with plate-bound anti-CD3 Ab along with control-Ig (Ctrl-Ig), DD1α-Ig, PD-1-Ig, or PD-L1-Ig as indicated in the figure. CD4⁺ or CD8⁺ T cells were incubated for 3 days before performing FACS analysis, and cytokine production by Wt or PD-1-null or DD1α-null T cells (IFN-γ and IL-2 levels in supernatants on day 2) was determined by ELISA (bottom). Data are shown as mean±SD and representative of three independent experiments

FIGS. 4A-1, 4A-2, 4A-3, 4A-4, 4B-1, 4B-2, 4B-3, 4B-4, 4B-5, 4B-6, 4B-7, 4B-8, 4B-9, 4B-10, 4C-1, 4C-2, 4C-3, 4C-4, 4C-5, 4C-6, 4C-7, 4C-8, 4C-9, 4C-10, 4C-11, 4C-12, 4C-13, 4C-14, 4C-15, 4C-16, 4C-17, 4C-18, 4C-19, and 4C-20. These figures show that the loss of DD1α expression in CD11b⁺Gr-1⁺ MDSCs enhances T cell proliferation. (A) Decreased binding of CD11b⁺GR-1⁺ MDSCs from DD1α-deficient mice to CD8⁺ T cells. To obtain bone marrow-derived CD11b⁺Gr-1⁺ MDSCs, bone marrow progenitor cells from Wt and DD1α^{-/-} mice were cultured in medium supplemented with different combinations of GM-CSF, G-CSF, IL-6, and 10% conditioned medium of B16 melanoma cells. CFSE-stained CD11b⁺Gr-1⁺ cells were incubated with Far Red stained Wt or PD-1^{-/-} CD8⁺ T cells. After 1 hour incubation, bindings between CD11b⁺Gr-1⁺ cells and CD8⁺ T cells were analyzed by counting the percentage of CFSE and Far Red positive populations. (B) PD-1 on T cells is required for CD11b⁺Gr-1⁺ MDSC-mediated T cell suppression. CD4⁺ and CD8⁺ T cells were isolated from Wt or PD-1^{-/-} mice, and 1×105 cells were seeded in 5 μg/ml α-CD3-coated 96 well plates with or without bone marrow-derived CD11b⁺Gr-1⁺ MDSCs. The proliferations of CD4⁺ or CD8⁺ T cells were determined by the percentage of T cells containing diluted CFSE signal on day 3. (C) Suppression of CD4⁺ and CD8⁺ T cell stimulation by CD11b⁺Gr-1⁺ MDSCs from Wt, DD1α^{-/-}, PD-L1^{-/-}, PD-1^{-/-}, and p53^{-/-} mice. DD1α expression was analyzed in cells as indicated (second from top). CFSE-labeled CD4⁺ or CD8⁺ T cells were stimulated by incubating them in α-CD3-coated plates for 72 hours, in the absence or presence of CD11b⁺Gr-1⁺ MDSCs isolated from spleens of D4M.3A-tumor bearing Wt, DD1α^{-/-}, PD-L1^{-/-}, PD-1^{-/-}, or p53^{-/-} mice. CFSE dilution in proliferation CD4⁺ or CD8⁺ T cells is depicted in each histogram (middle panels) and summarized by the bar graph (bottom). Data are representative of three independent experiments and values are expressed in mean±SEM.

FIGS. 5A, 5B-1, 5B-2, 5C-1, 5C-2, 5C-3, 5C-4, 5C-5, and 5C-6. These figures show an example of the intercellular heterotypic interaction between DD1α and PD-1. (A) Schematic of intercellular DD1α-PD-1 interaction. (B) The exogenous expression of DD1α in 293E cells and PD-1 in 293A cells was validated by flow cytometry analysis. (C) CFSE-stained 293E/DD1α cells were pre-incubated with Ig protein (control Ig: 40 μg/ml; PD-1-Ig: 20 μg/ml; PD-1-Ig: 40 μg/ml) for 30 min and mixed with Far Red-stained 293T control cells or Far Red-stained 293A/PD-1 cells. After 1 hour incubation, DD1α-PD-1 intercellular bindings were analyzed by counting the percentage of CFSE- and Far Red-positive populations.

FIGS. 6A and 6B. These figures show that incubation with anti-human DD1α monoclonal antibodies established in this study mediates increased T cell proliferation. Anti-DD1α monoclonal Abs were incubated with purified CD4⁺ T cells (top panel) and CD8⁺ T cells (bottom panel), along with 5 μg/ml CD3-specific Ab and 10 μg/ml DD1α-Ig. The three leftmost bars indicate T cell incubations with control Ig alone, CD3-specific Ab alone, or CD3-specific Ab+DD1α-Ig. The proliferations of CD4⁺ or CD8⁺ T cells were determined by percentage of CFSE-diluted CD4⁺ T cells on day 5. Data represent mean±SD from three different experiments.

FIGS. 7A and 7B. These figures show that treatment of Human PBMCs with anti-DD1α monoclonal Abs enhances IFN-γ and IL-2 production by normal human PBMCs. Human PBMCs were stimulated with 100 ng/ml CD3-specific Ab alone (Mock) or together with anti-DD1α monoclonal Abs, PD-1 Ab, or control Ig as indicated. IFN-γ and IL-2 levels in culture supernatant on day 3 were analyzed by ELISA. Graphs represent mean±SD of three experiments.

FIGS. 8A and 8B. (A) The nucleotide sequences and amino acid of the heavy chain variable region of the 2B11 human monoclonal antibody. (B) The nucleotide sequences and amino acid of the light chain variable region of the 2B11 human monoclonal antibody.

FIGS. 9A and 9B. (A) The nucleotide sequences and amino acid of the heavy chain variable region of the 2E2 human monoclonal antibody. (B) The nucleotide sequences and amino acid of the light chain variable region of the 2E2 human monoclonal antibody.

FIGS. 10A and 10B. (A) The nucleotide sequences and amino acid of the heavy chain variable region of the 4D5 human monoclonal antibody. (B) The nucleotide sequences and amino acid of the light chain variable region of the 4D5 human monoclonal antibody.

FIGS. 11A and 11B. (A) The nucleotide sequences and amino acid of the heavy chain variable region of the 4H8 human monoclonal antibody. (B) The nucleotide sequences and amino acid of the light chain variable region of the 4H8 human monoclonal antibody.

FIGS. 12A and 12B. (A) The nucleotide sequences and amino acid of the heavy chain variable region of the 5C2 human monoclonal antibody. (B) The nucleotide sequences and amino acid of the light chain variable region of the 5C2 human monoclonal antibody.

FIGS. 13-1, 13-2, 13-3, 13-4, 13-5, and 13-6. These figures show that the binding of DD1α and PD-1 to DD1α overexpressed Jurkat cells was diminished upon treatment with anti-DD1α monoclonal Abs. Jurkat/DD1α cells were pre-incubated for 30 min with 15 μg/ml DD1α-specific Ab 2B11 or 5C2, or with control IgG1 Ab. Cells were assayed for protein bindings by staining with DD1α-Ig (blue open), PD-1-Ig (red open) or control Ig proteins (gray filled). The bound Ig fusion proteins were detected by Ab against Ig-PE. Binding amounts were shown by percentage of fluorescence-positive cells compared with control Ig protein-bound cells.

FIGS. 14-1, 14-2, 14-3, 14-4, and 14-5. These figures show the CD11b⁺Gr1^{high} MDSC population isolated from Wt, DD1α^{-/-}, PD-L1^{-/-}, PD-1^{-/-} and p53^{-/-} tumor bearing mice. Cells were analyzed with Gr-1-PE and CD11b-APC by flow cytometry.

FIGS. 15-1, 15-2, 15-3, and 15-4. These figures show the protein expression profiles of DD1α and PD-L1 in a variety of human cancer cell lines and normal cells with several different origins. Cell lysates were analyzed by Western blotting for the levels of DD1α and PD-L1, with β-actin used as loading control.

FIGS. 16A, 16B, and 16C. These figures show the RNA expression profiles of DD1α, PD-L1 and PD-L2 in a variety of human cancer samples.

FIG. 17 shows the process for preparing BM-derived CD11b⁺Gr-1^{high}Ly6G⁺ MDSC cells. CD11b⁺Gr-1^{high}Ly6G⁺ MDSC cells were positively selected with Ly-6G antibody by magnetic-activated cell sorting.

FIG. 18 shows a summary for personalized therapeutic and diagnostic validation of the immune checkpoint pathway for the MDSC-mediated suppression in cancer patients.

FIGS. 19-1 and 19-2. These figures show apoptosis in CPT-treated thymocytes isolated from Wt-mice as determined by Annexin V/PI staining.

FIG. 20. Table 1. Identification of potential DD1α/VISTA-interacting proteins by mass spectrometry analysis (Excel files, transmembrane proteins selected from the file).

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The terms “percent identical” or “percent identity” refer to nucleic acid or amino acid sequences that are substantially identical to a coding sequence or amino acid sequence including the amino acid sequences of the isolated DD1α antigen encoded by SEQ ID NO. 1, the wild type DD1α and PD-1 proteins encoded by SEQ ID NOs 2 and 3, or the heavy and light chains (SEQ ID NOs: 4-13) for the isolated monoclonal antibodies described herein.

The term “substantially identical” means nucleotide sequence with similarity to the nucleotide sequence (SEQ ID NOs: 14-23) of the isolated monoclonal antibodies described herein. The term “substantially identical” can also be used to describe similarity of polypeptide sequences. For example, nucleotide sequences or polypeptide sequences that are at least 70%, 75%, 80%, 85%, 90%, 92%, 95%, 96%, 98% or 99% identical to the coding sequences (SEQ ID NOs: 14-23), or the encoded polypeptides thereof (SEQ ID NOs: 4-13), respectively, or fragments or derivatives thereof, and still retain ability to bind to the DD1α peptide (SEQ ID No. 1). The term “substantially identical” additionally refers to polypeptide sequences that are at least 70%, 75%, 80%, 85%, 90%, 92%, 95%, 96%, 98% or 99% identical to the isolated DD1α antigen (SEQ ID NO 1) or the wild type DD1α or PD-1 proteins (SEQ ID NOs. 2 and 3), or fragments or derivatives thereof, provided that the isolated monoclonal antibodies can still bind to the DD1α antigen and disrupt the interaction between the wild type DD1α and PD-1 proteins, fragments or derivatives thereof.

With references to the sequences described herein, SEQ ID NO 1 represents the amino acid sequence of the isolated human DD1α antigen used to generate the isolated monoclonal antibodies described herein. SEQ ID NO 2 represents the amino acid sequence for the wild type human DD1α protein to which the isolated monoclonal antibodies bind. SEQ ID NO 3 represents the amino acid sequence for the wild type human PD-1 protein. The interaction of the PD-1 protein with the DD1α protein is disrupted upon binding of the isolated monoclonal antibodies described herein to the DD1α protein. SEQ ID NOs 4-13 represent the amino acid sequences of the isolated monoclonal antibodies described herein. SEQ ID NOs 14-23 represent the nucleotide sequences for the isolated monoclonal antibodies described herein.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

It should be appreciated that the nucleotide and amino acids sequences provided herein may or may not be isolated sequences.

Throughout this application, where publications are referenced, the disclosures of these publications in their entireties are hereby incorporated by reference into this application to more fully describe the state of the art to which this invention pertains.

### Abbreviations

MDSC is an acronym for myeloid-derived suppressor cells
mAb is an abbreviation for monoclonal antibody
Wt is an abbreviation for wild type
PD-1 is an abbreviation for Programmed Death-1
DD1α is an abbreviation for Death Domain 1α

Described are methods and compositions for treatment of immune-related diseases/disorders by modulating DD1α activity, alone or in combination with modulation of PD-1 and/or PD-L1 activity. Of importance, through proteomic and cell-based experiments, this disclosure offers the receptor DD1α as a novel intercellular binding partner of the PD-1 immune-checkpoint receptor. The novel use of DD1α's role as a negative regulator of T cell activation is dependent on an inhibitory bidirectional ligand/receptor interaction between DD1α and PD-1. Disruption of the DD1α-PD-1 pathway by DD1α antagonist(s)/modulator(s), preferably antibodies, should enhance anti-tumor immunity by T-cell activation. The present invention relates to the identification of a novel regulatory DD1α/PD1 immune receptor pathway, in which DD1α serves as a heterotypic ligand or receptor for the PD-1 checkpoint regulator. The physical interaction between DD1α and PD-1 is required for the ability of DD1α to mediate T cell suppression.

Additionally, high expression of the DD1α receptor in CD11b⁺Gr-1⁺ myeloid-lineage cells (defined as myeloid-derived suppressor cells, MDSCs) is critical for the direct suppression of T-cell activation. In this way, the DD1α-PD-1 axis serves as an alternative T cell checkpoint pathway that is exploited by MDSCs during cancer progression anti-tumor immunity. Targeting this new arm of the PD-1 pathway may therefore increase the efficacy of therapeutic strategies to modulate anti-tumor immunity. The expression of negative immune checkpoint regulators by cancer cells or immune cells in the tumor microenvironment can suppress the host's immune response against the tumor. To effectively combat the cancer, it is beneficial to impede tumor-mediated suppression of the host immune response. Hence, there is an emerging demand for new and effective therapeutic agents that inhibit negative immune regulators in the tumor microenvironment that suppress anti-tumor immune responses. In some good examples here, the methods and compositions described herein are mainly directed to treatment of cancer and also to infections (e.g., bacterial infection, viral infection, and/or fungal infection). Methods for identifying patients who are more likely to be responsive to and benefit from a cancer immunotherapy that targets the DD1α-PD-1 pathway or expression of these proteins are also described herein. The disclosure also provides monoclonal antibodies (mAbs) and their sequences, which bind specifically to a cell surface-expressed DD1α antigen, and disrupt the association between DD1α and PD-1 receptors. The present application relates to antibodies specifically binding to the IgV-domain of DD1α for their use in cancer treatment.

Also, provided herein are compositions and methods based, at least in part, on the discovery that there is a T cell and macrophage signaling axis involving tumor suppressor p53, DD1α and PD-1/PD-L1. In one aspect, the inventors have identified the DD1α receptor as a post-apoptotic target gene of Wt tumor suppressor p53, which is induced in apoptotic cells and highly expressed in immune cells, e.g., but not limited to macrophages, dendritic cells, monocytes, myeloid cells and T cells. Further, the inventors have shown that the expression of three B7 family members, DD1α, PD-1 and PD-L1, is significantly induced in response to chemotherapeutic drugs or radiation in human cancer cells containing wild type (Wt) p53 in a p53-dependent manner. Because the DD1α receptor is known to function as a negative immune checkpoint regulator/inhibitor that is involved in modulating immune response and/or T cell function, the inventors claim that the co-treatment strategy combining DD1α modulators (such as DD1α blocking antibodies) and chemo-therapeutic agents or radiation will be more effective in combating DD1α-expressing cancers as compared to conventional chemo- and/or radiation therapy.

The discovery additionally provides a method for cancer immunotherapy, which involves administering to the cancer patient an antibody that inhibits signaling from the DD1α/PD-1 pathway, the DD1α/DD1α pathway, or a combination of such antibodies, and/or tumor specific delivery of the therapeutic anti-DD1α antibody proteins via methods commonly known in the art such as engineered adeno-associated virus (AAV) vectors.

The present invention disclosure also provides a method for immunotherapy of cancer patients which includes identifying patients who are suitable candidates for immunotherapy, by assessing whether the fraction of cells in tumor tissue samples from the patient(s) that express DD1α on the cell surpasses a decided threshold level as compared to a reference sample, and administering an anti-DD1α Ab to the patient(s). The disclosure additionally provides monoclonal antibodies that bind specifically to a cell surface-expressed DD1α antigen in a cancer tissue sample, and an Immunohistochemistry method for assessing cell surface expression in tissue samples using the offered anti-DD1α Abs.

The term “immunotherapy” as described herein, refers to the treatment of a disease or disorder including cancer by modulating the regulatory activity of the components of the DD1α-PD-1 immune regulatory checkpoint signaling axis. For example, modulating the regulatory activity of the components of the DD1α-PD-1 axis can include activating or inhibiting the DD1α or PD-1 proteins; disrupting the intercellular physical interaction between DD1α and PD-1 or between two DD1α proteins; or altering the function intercellular interaction between DD1α-PD-1 or between two DD1α proteins.

The term “modulating” as used herein means to alter the expression or activity of a target molecule such as a protein. As known to a person skilled in the art, modulating generally means increasing or decreasing expression of a target molecule, or enhancing, inhibiting or altering a known activity of that target molecule. For example, DD1α activity can be modulated by a treatment that increases or reduces the level of DD1α protein present in a cell or sample. Modulation of DD1α activity can also include treatments that enhance, disrupt or alter the binding of the DD1α protein to its targets, which can include binding partners, ligands, substrates and the like. Modulation of DD1α activity can further be achieved by any treatment that alters the molecular structure, composition, function or activity of not only the DD1α protein itself, but also of any of its binding partners, ligands, substrates and the like, including any molecules that function in any biological or physiological pathways in which the DD1α protein is involved.

The effects of modulating a target can be measured by any suitable in vitro, in vivo or cellular assay, using any measurement techniques commonly used in the art. Such techniques can include, for example, fold change increases or decreases in target expression in the treated sample as compared to a sample without the treatment that causes the target expression to change; percentage increase or reduction in binding of the target molecule to itself, a binding partner, a ligand, a substrate or the like; change in binding avidity or affinity measure in terms of a difference in dissociation (K_{D}) and/or association (K_{A}) constants in a treated versus an untreated sample; or similar measurement methods commonly utilized in the art.

The term “threshold” as used herein refers to the level of expression of a target molecule beyond which a biological or physiological equilibrium is shifted, leading to changes in biological or physiological activity. In some embodiments, when the level of DD1α expression on tumor-derived MDSC cells in a sample obtained from a patient exceeds an identified threshold as compared to a sample from a patient with no tumor, the patient with the tumor will experience a decrease in T-cell proliferation and consequently a reduction in anti-tumor immunity. This decrease will lead to a poor prognosis, as the tumor will continue to grow. The threshold can be identified by using any suitable expression measurement techniques known in the art including RNA expression measurement techniques such as reverse transcriptase PCR or next generation sequencing techniques, and the like; protein expression measurement techniques including ELISA-based techniques, Western blotting, flow cytometry techniques including fluorescence activated cell sorting (FACS), and the like.

In some embodiments, the threshold can be identified as follows: Bodily fluid or tissue samples (e.g but not limited to blood, tissue biopsy, and the like) can be obtained from a group of patients experiencing cancer. Similar samples can be obtained from patients with no cancer as a reference sample. The average expression level of the target molecule (e.g but not limited to DD1α) in the samples obtained from cancer patients can be compared to the average expression level of the target molecule in the samples obtained from patients without cancer. A measure of the biological or physiological activity affected by the change in target molecule expression can also be compared between cancer patient and normal patient samples. For example, in some embodiments, T cell proliferation can be measured in both groups of patients. The threshold of target molecule (e.g but not limited to DD1α) expression can be identified as the expression level at which T cell proliferation is affected (e.g but not limited to the DD1α expression level associated with reduced T cell proliferation). In some embodiments, the increase in DD1α expression that correlates with reduced T cell proliferation can be at least about 10% or more, including at least about 20%, at least about 30%, at least about 40%, at least about 50%, at least about 60%, at least about 70%, at least about 80%, at least about 90%, at least about 95% or more. The corresponding reduction in T cell proliferation can be at least about 10% or more, including at least about 20%, at least about 30%, at least about 40%, at least about 50%, at least about 60%, at least about 70%, at least about 80%, at least about 90%, at least about 95% or more. In other embodiments, the increase in DD1α expression that correlates with reduced T cell proliferation can be at least about 1.1 fold or more, including at least about 2-fold, at least about 3-fold, at least about 4-fold, at least about 5-fold, at least about 6-fold, at least about 7-fold, at least about 8-fold, at least about 9-fold, at least about 10-fold, at least about 25 fold, at least about 50-fold, at least about 100-fold or more. The corresponding reduction in T cell proliferation can be at least about 1.1 fold or more, including at least about 2-fold, at least about 3-fold, at least about 4-fold, at least about 5-fold, at least about 6-fold, at least about 7-fold, at least about 8-fold, at least about 9-fold, at least about 10-fold, at least about 25 fold, at least about 50-fold, at least about 100-fold or more.

In some embodiments, expression levels of the DD1α protein are measured on MDSC cells isolated from bodily fluid or tissue samples (e.g but not limited to blood, tissue biopsy, and the like) from patients experiencing cancer. The expression levels can be measured by protein expression measurement techniques known in the art including immunohistochemistry, ELISA-based or flow cytometric techniques, and the like, utilizing at least one of the mAbs described herein, the heavy and light chains comprising SEQ ID NO. 4 and 5, respectively, SEQ ID NO. 6 and 7, respectively, SEQ ID NO. 8 and 9, respectively, SEQ ID NO. 10 and 11, respectively, and SEQ ID NO. 12 and 13 respectively.

The term “anti-cancer therapeutic” as used herein generally means an agent that kills cancer cells, and/or slows the proliferation of cancer cells, and/or slows or prohibits tumor growth and/or progression and/or metastasis. Anti-cancer therapeutics can include any anti-cancer therapies commonly known to those skilled in the art, including but not limited to, chemotherapy, radiation therapy, immunotherapy, targeted therapies (e.g. kinase, angiogenesis, or proteasome inhibitors), surgery, and combinations thereof.

The term “reference sample” as used herein signifies a sample whose value can be used as a reference level during comparison. For example, the reference sample can be used to determine whether a treatment effects a change in a particular measurement by comparing the measurement in the treated sample versus the reference sample. In some embodiments, the reference sample refers to the average level of DD1α expression measured across bodily fluid or tissue samples obtained from patients with no detectable form of cancer.

The term “monoclonal antibody” as used herein refers to an antibody derived from a population of antibodies in which the antibodies recognize a single epitope on an antigen, and are identical to each other but for naturally occurring mutations that may occur with low frequency. Monoclonal antibodies may be generated by various methods commonly known in the art, such as the hybridoma method (Kohler et al., Nature 256:495 (1975)), recombinant DNA methods (U.S. Pat. No. 4,816,567), and/or phage display methods (McCafferty et al., Nature 348:552 (1990)).

In some embodiments, the monoclonal antibodies described herein may include derivatives that are modified. The antibodies can be modified by any covalent modification that does not disrupt the binding of the antibody to the DD1α antigen encoded by SEQ ID No. 1, and that does not affect the ability of the antibody to disrupt the intercellular physical interaction between DD1α and DD1α or PD-1. Modifications can include but are not limited to pegylation, phosphorylation, acetylation, glycosylation and the like, and/or combinations thereof.

In some embodiments, the amino acid sequences of the antibodies described herein can be modified. Deletions, insertions or substitutions can be made to the amino acid sequences by either introducing such changes into the nucleic acid sequence encoding the antibody or by peptide synthesis. The described changes can alter any biological properties of the antibodies, including binding affinity or post-translational modification or processing of the antibody protein, provided that the modified antibodies still bind to DD1α/DD1α or DD1α/PD-1 specifically.

The composition described herein can be delivered to a patient experiencing an immune related disease or disorder, including but not limited to cancer, by any suitable methods commonly known in the art. Non-limiting examples include infusion, intravenous, subcutaneous, intramuscular or intradermal injection, and the like. The antibody composition can be delivered by these methods along with a pharmaceutically acceptable carrier or vehicle, including but not limited to, sterile water, saline solution, dextrose solution, polyethiene glycol, etc.

The term “pharmaceutically acceptable” as used herein refers to compositions, materials, compounds or dosage forms that, according to sound medical judgment, are suitable for use in, on, or around human or animal tissues with minimal instance of undesired effects including toxicity, irritation, allergic response, or the like.

The dosage range for the monoclonal antibody composition described herein includes amounts capable of producing the desired effect, without introducing undue side effects. The dosage can be determined by a person skilled in the art, and can be adjusted by prescribing physicians in the event of complications. The dosage range depends on the biological and physiological characteristics of the patient, including but not limited to age, sex, weight, or condition. The therapeutically effective dose, which is the amount of the composition required to achieve a statistically significant change in a symptom to be treated, can be determined in animal studies and clinical trials. In some embodiments, the therapeutically effective dose can be the amount of the composition described herein required to induce a statistically significant increase in T cell proliferation in a patient experiencing a cancer in which MDSC's express DD1α on the cell surface.

The term “treatment” or “treated” as described herein refers to prophylaxis or therapy, including but not limited to administration of the antibody composition described herein to a patient or subject via infusion, injection or the like. Treatment can occur by a single infusion or injection at a single time point, or by multiple infusions or injections over multiple time points. Patients or subjects that can be treated include humans, but can also include non-human mammals including veterinary subjects.

The term “cancer” as used herein applies to an uncontrolled growth of abnormal cells that is able to infiltrate and destroy normal body tissue and interfere with normal biological and physiological functions in a patient or subject. A patient or subject experiencing cancer is a patient or subject having a measurable amount of cancer cells in the patient or subject's body.

The immunoglobulin superfamily is composed of numerous important immune regulators, including those of the B7 family. In general, the B7 family is composed of a group of structurally-related protein ligands, expressed on the surface of both lymphoid and non-lymphoid cells. B7 family ligands are known to deliver both co-stimulatory and co-inhibitory signals to lymphocytes via the CD28 family of receptors. V-domain Ig suppressor of T cell activation (VISTA), which is also known as PD-1H or DD1α, is a critical member of the B7 family that is important for regulating autoimmunity and immune surveillance. DD1α is expressed on the surface of hematopoetic cells, including antigen presenting cells (APCs), CD4⁺ and CD8⁺ T cells, natural killer (NK) cells, macrophages, dendritic (DC) cells and neutrophils. DD1α expression is increased within the tumor microenvironment in many different types of cancer, where it inhibits anti-tumor immunity. The inventors discovered that a specific intercellular interaction between DD1α on tumor infiltrating myeloid cells such as MDSCs and DD1α or PD-1 on T cells is important in promoting the DD1α-mediated inhibition of anti-tumor immunity. Provided herein are monoclonal antibodies that bind specifically to the DD1α antigen represented by SEQ ID NO. 1. These mAb disrupt the intercellular interaction between DD1α on the surface of a myeloid cell, including an MDSC, and a second DD1α molecule or a PD-1 molecule on the surface of a T cell, including CD4⁺ or CD8⁺ T cells.

DD1α, as used herein, refers to either the wild type DD1α protein having the amino acid sequence of SEQ ID NO. 2, and any naturally occurring variants of this sequence. DD1α also refers to polypeptides, modified or unmodified, with a sequence having at least about 70% sequence identity to the sequence of SEQ ID NO. 2. For example, the polypeptide can have a sequence of at least about 70%, or at least about 75%, or at least about 80%, or at least about 85%, or at least about 90%, or at least about 95%, or more sequence identity to SEQ ID NO. 2.

Programmed death 1 (PD-1) functions as a receptor on T-cells and downregulates T-cell receptor-induced signals to promote self-tolerance and inhibit autoimmunity. Through this same activity, PD-1 has also been shown to prevent T-cell activation in tumors, thereby inhibiting anti-tumor immunity. Therapies blocking the PD-1/PD-1L axis have shown promise in promoting anti-tumor immunity. Despite some success, however, these therapies have proven to be susceptible to a significant degree of resistance. One possible mechanism of resistance is that these therapies do not target the intercellular interaction between DD1α molecules or between DD1α and PD-1. The inventors have discovered that this interaction is critical for the inhibition of T-cell proliferation mediated by the DD1α-PD-1 axis. The anti-DD1α mAb treatment provided herein disrupts the intercellular interaction between DD1α on myeloid cells such as MDSCs, and PD-1 on T cells, including CD4⁺ and CD8⁺ T cells. Disrupting this interaction provides a novel method for bypassing resistance mechanisms inherent to current PD-1/PD-1L blockade therapies, and likely provides an additional therapy that can promote anti-tumor immunity.

PD-1, as used herein, refers to either the wild type PD-1 protein having the amino acid sequence of SEQ ID NO. 3, and any naturally occurring variants of this sequence. PD-1 also refers to polypeptides, modified or unmodified, with a sequence having at least about 70% sequence identity to the sequence of SEQ ID NO. 3. For example, the polypeptide can have a sequence of at least about 70%, or at least about 75%, or at least about 80%, or at least about 85%, or at least about 90%, or at least about 95%, or more sequence identity to SEQ ID NO. 3.

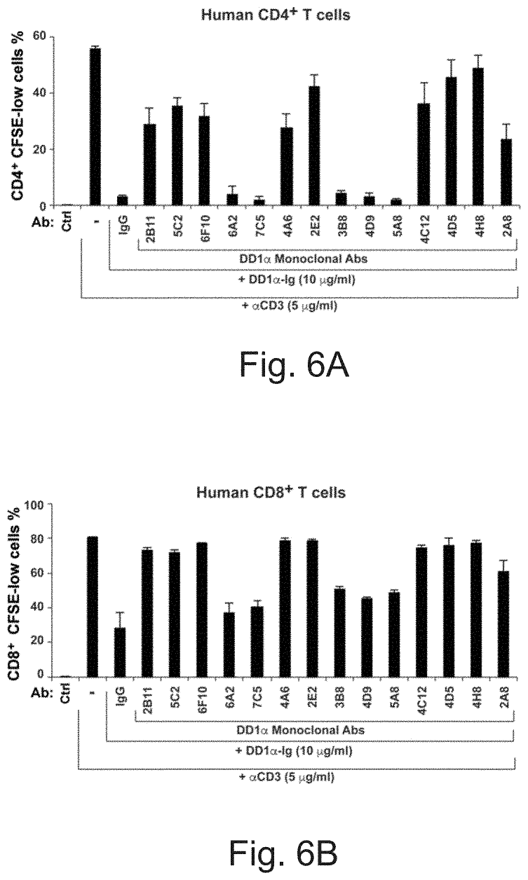

The hybridoma lines generated and used to produce the antibodies described herein are detailed in FIGS. 6 &7. The names of the monoclonal antibodies represent the hybridoma lines.

The following examples illustrate the various embodiments of the present invention. Those skilled in the art will recognize many variations that are within the spirit of the present invention and scope of the claims.

A high level of cell death occurs within tumors during cancer therapy, and the mechanisms through which dying tumor cells are cleared influence anti-tumor immunity. The inventors previously identified a post-apoptotic p53 target gene, DD1α/VISTA/PD-1H which appears to function as an immunoregulator of T cell tolerance. DD1α facilitates the phagocytosis of apoptotic cell bodies and further links to the priming of immune tolerance. Thus, VISTA/DD1α links cell death with tolerance to self-antigens. Through the following examples, the inventors demonstrate that VISTA/DD1α's role as a negative regulator of T cell activation is in part dependent on the immune checkpoint regulator, PD-1, indicating a novel inhibitory bidirectional ligand/receptor interaction between VISTA/DD1α and PD-1. The DD1α-PD-1 pathway potently inhibits T-cell activation. Moreover, DD1α in CD11b⁺Gr-1⁺ myeloid-lineage cells (defined as myeloid-derived suppressor cells, MDSCs) is critical for the direct suppression of T-cell activation. Together the data suggest that DD1α serves as a heterotypic ligand or receptor for the PD-1 checkpoint regulator that is required for its ability to mediate T cell suppression. The inventors thus demonstrate that the DD1α-PD-1 axis is an alternative T cell checkpoint pathway that is exploited by MDSCs during cancer progression, suppressing anti-tumor immunity. They propose that targeting this new arm of the PD-1 pathway may increase the efficacy of therapeutic strategies to modulate anti-tumor immunity.

Throughout the lifetime of an organism, numerous cells undergo cell death either during normal development or in response to stress or injury, demanding rapid and efficient clearance of cell corpses as a prerequisite for the maintenance of tissue integrity and function (1-7). Failure to clear dying cells can lead to the accumulation of autoantigens in tissues that foster diseases such as chronic inflammation, autoimmunity, and developmental abnormalities (1, 4, 6, 8, 9). Clearance of apoptotic cells is accompanied by induction of immune tolerance through the activation of immune checkpoint pathways, which prevents self-antigen recognition (3, 7, 10-13). Thus, immune checkpoint regulators have evolved to regulate the activation of immune cells and prevent their hyperactivation which would impair normal host physiology. The CTLA-4 receptor and PD-L1/PD-1 axis are two of the best-characterized immune checkpoint pathways that play principal roles in regulating both central and peripheral T cell tolerance (12, 14-22).

Several recent studies have demonstrated that VISTA/DD1α (also referred to as PD-1H) is expressed on T cells and can function as a co-inhibitory receptor of T cell activation, similar to CTLA-4 and PD-1 (23-28). The inventors previously showed that p53 induction of DD1α is an essential step in ensuring proper clearance of dead cells, potentially leading to immune tolerance (13, 23, 29). Concordant with these results, they observed that systemic DD1α deficiency ultimately leads to immune dysfunction and breakdown of self-tolerance in mice (23). However, the mechanism of DD1α-mediated T cell suppression remains poorly understood. Studies to date have not eliminated the possibility that DD1α may act as a ligand or receptor for cell surface molecules other than itself. The following examples therefore summarize the inventor's investigation into the molecular interactions that underpin DD1α's ability to regulate T cell responses.

### Example 1: DD1α is Involved in Mediating T Cell Tolerance

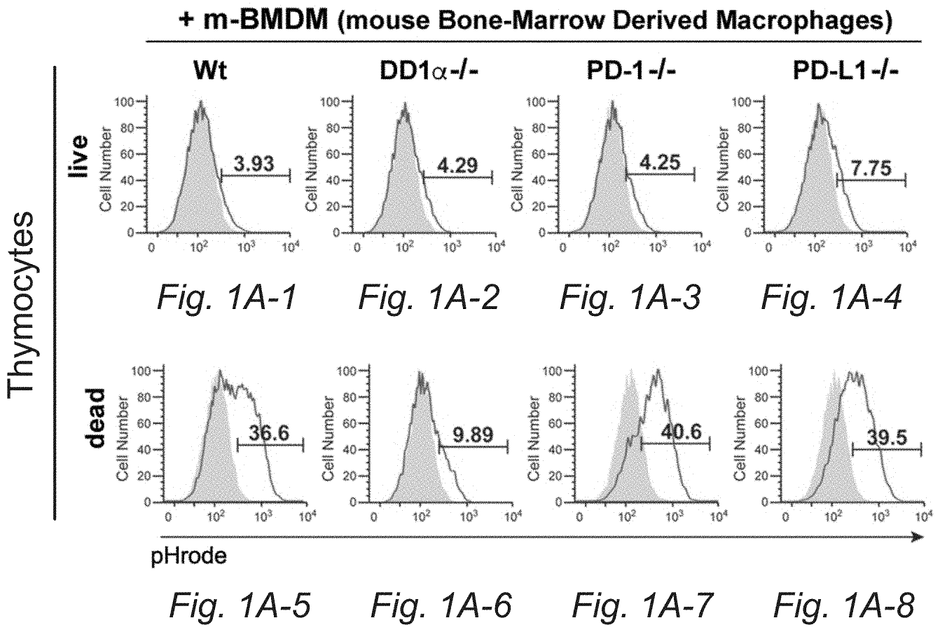

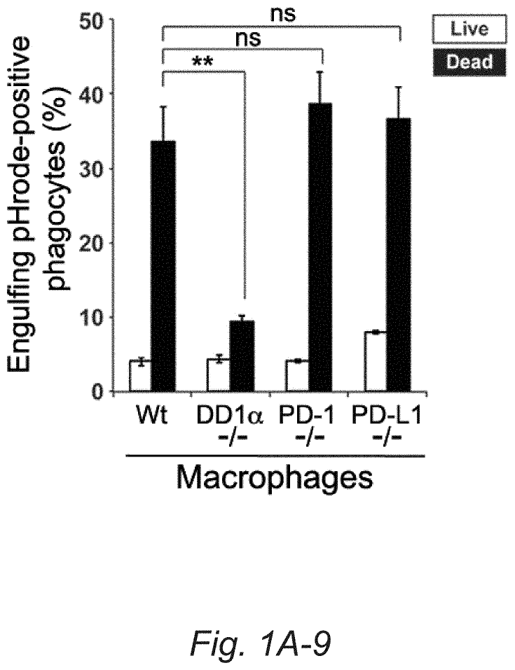

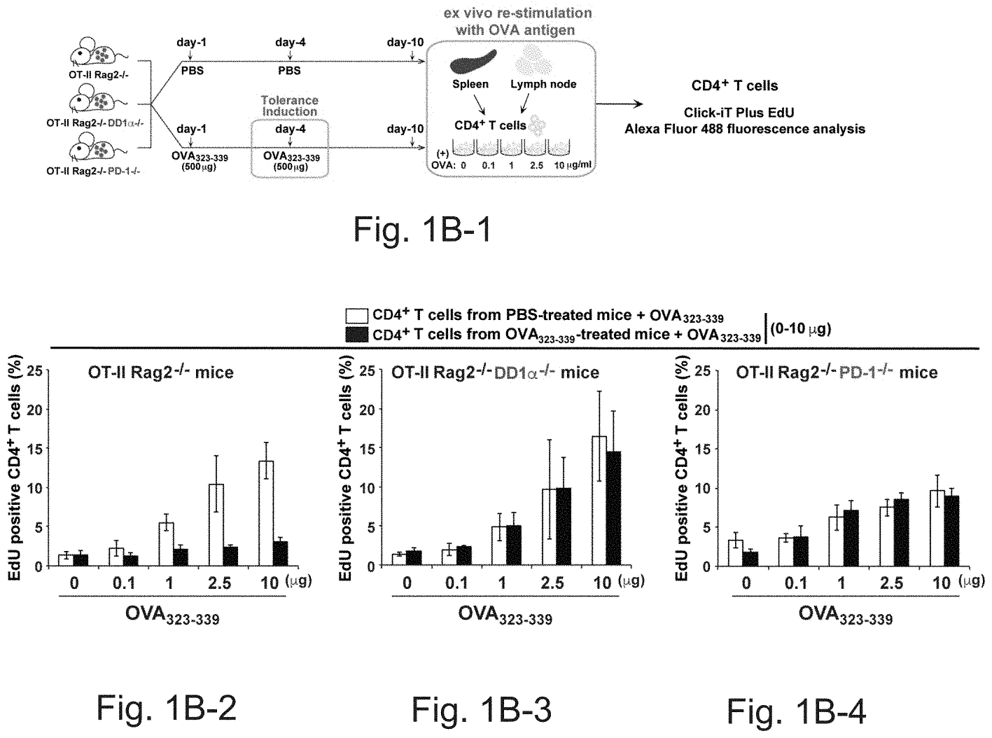

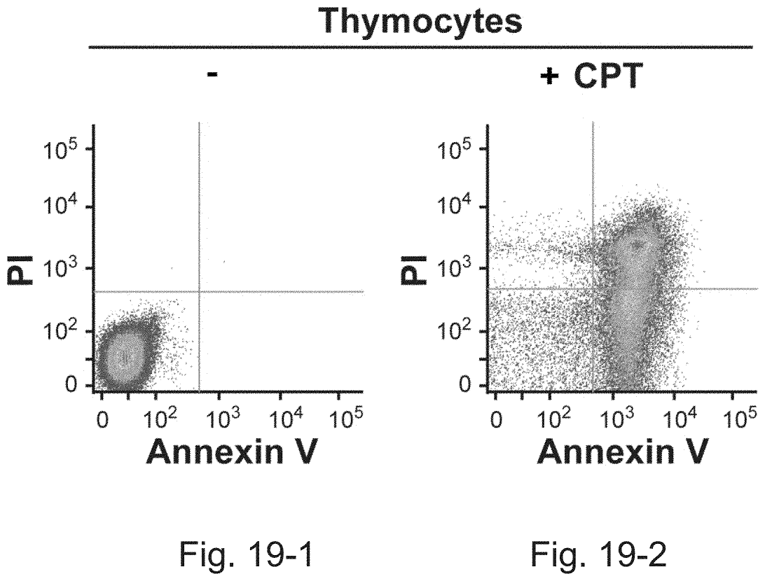

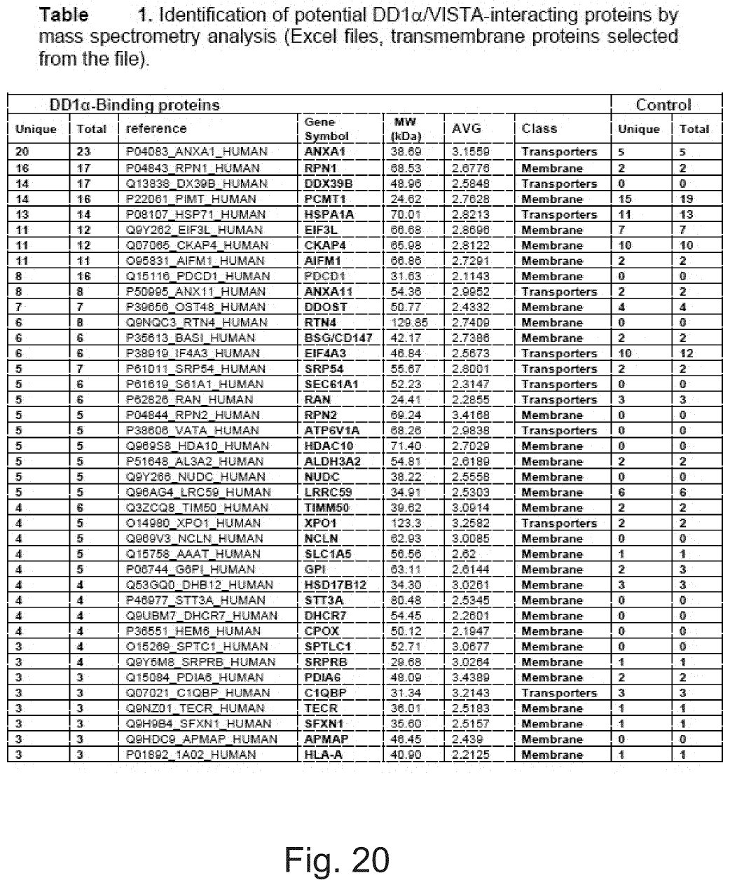

Given that DD1α regulates the engulfment of apoptotic cells (23), the inventors first investigated if the PD-1 co-inhibitory receptor and its ligand PD-L1 function as scavenger receptors, and if their expression on macrophages is also necessary for the engulfment of apoptotic cells. They isolated thymocytes from Wt mice and treated them with camptothecin (CPT) to induce apoptosis (FIG. 19), and then incubated them with bone marrow-derived macrophages (m-BMDM) isolated from PD-1^{-/-}, or PD-L1^{-/-} mice. m-BMDMs with loss of PD-1- or PD-L1 did not appear to have any reduction of phagocytic ability when compared to Wt m-BMDMs, showing similar rates of apoptotic thymocyte engulfment, unlike m-BMDMs with loss of DD1α (FIG. 1A), indicating that neither PD-1 nor PD-L1 regulates engulfment of apoptotic cells.

The inventors next utilized a model system that mimics T cell tolerance (30, 31) to investigate the role of DD1α in OVA-specific peripheral T cell tolerance in vivo. OVA-specific T cell receptor transgenic OT-II Rag2^{-/-} mice were immunized with chicken OVA₃₂₃₋₃₃₉ peptide antigen and tolerance to the peptide was induced as previously shown (30) (schematized in FIG. 1B). OVA peptide re-stimulation of CD4⁺ T cells isolated from OVA peptide-treated OT-II Rag2^{-/-} mice revealed strong tolerance, while CD4⁺ T cells from PBS-treated OT-II Rag^{-/-} mice proliferated in response to OVA peptide challenge (FIG. 1B). To next evaluate DD1α-induced T cell tolerance, the inventors crossed DD1α^{-/-} mice or PD-1^{-/-} mice with the OT-II Rag2^{-/-} mice. Loss of either DD1α or PD-1 in CD4⁺ T cells from OVA peptide-tolerized OT-II Rag2^{-/-} mice rescued OVA peptide-induced proliferation of those CD4⁺ T cells, to levels similar to OVA peptide-mediated proliferation of CD4⁺ T cells from PBS-treated OT-II Rag2^{-/-} DD1α^{-/-} mice or OT-II Rag2^{-/-} PD-1^{-/-} mice (FIG. 1B). Thus, in vivo loss of DD1α in TCR transgenic OT-II CD4⁺ T cells prevents the induction of T cell tolerance to a peptide antigen. DD1α expression equivalently restricted to proliferating DD1α⁺ T cells in vivo. Moreover, DD1α has been proposed to engage in immune checkpoint regulation through an unknown receptor/ligand (24, 25) although DD1α appears to function as both a ligand and receptor of T cell suppression by forming homotypic intercellular interactions (23).

### Example 2: Identification of PD-1 Receptor as a Novel DD1α Binding Partner

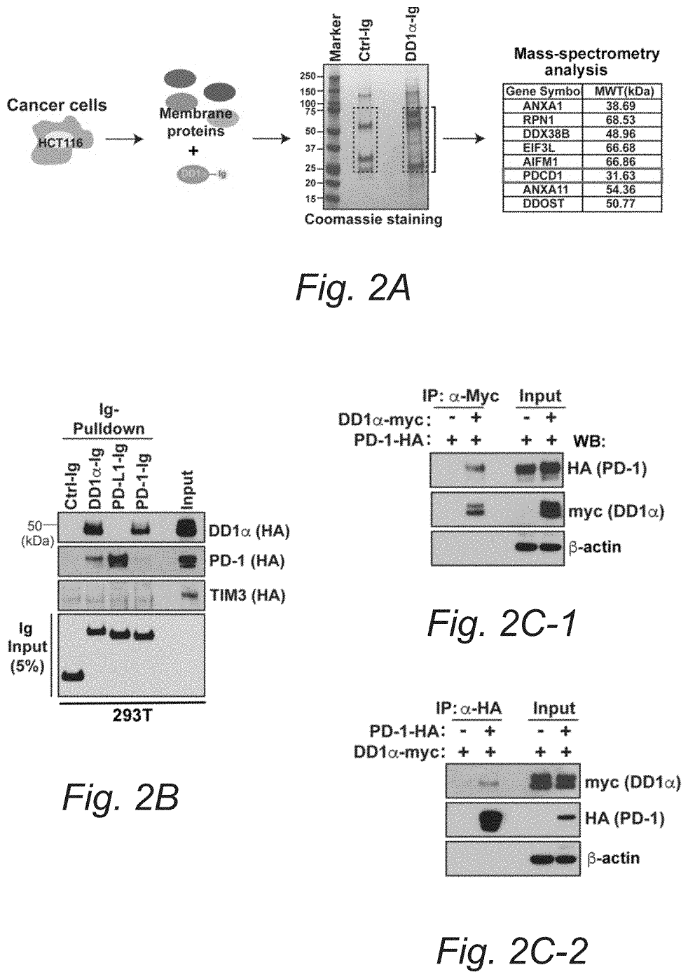

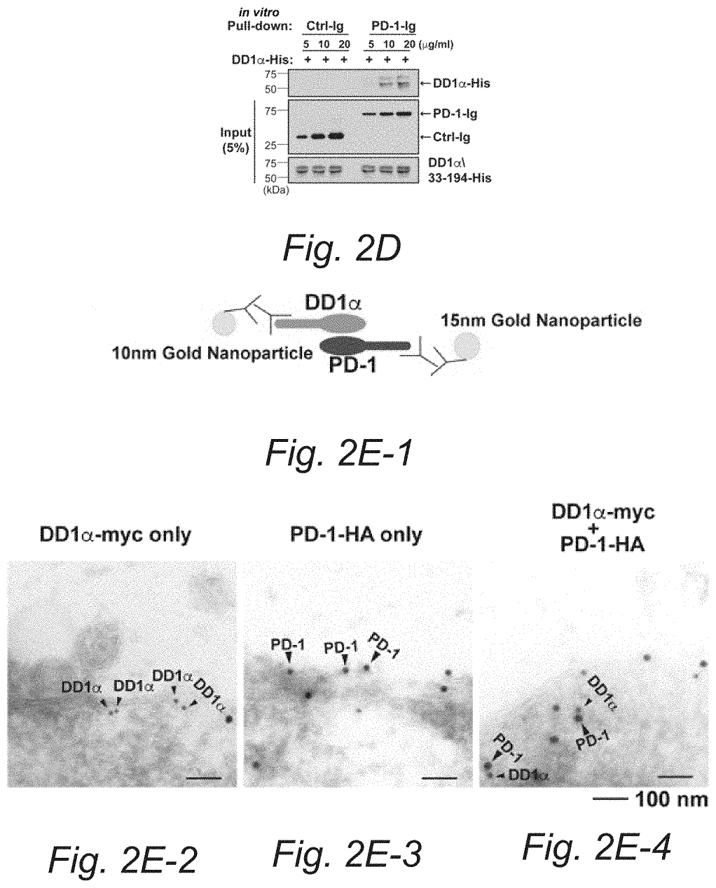

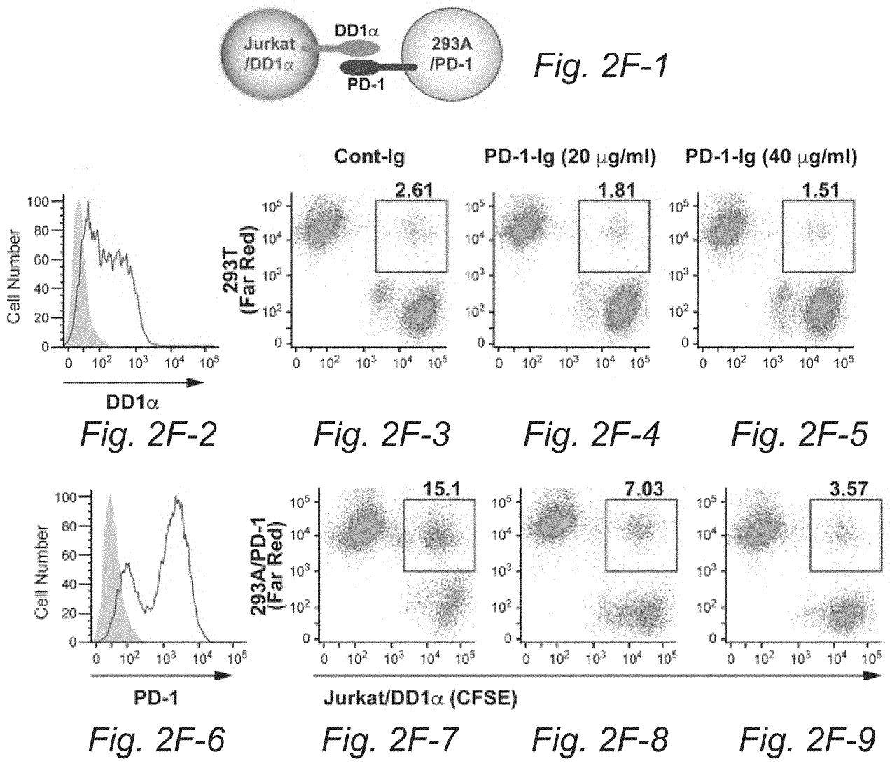

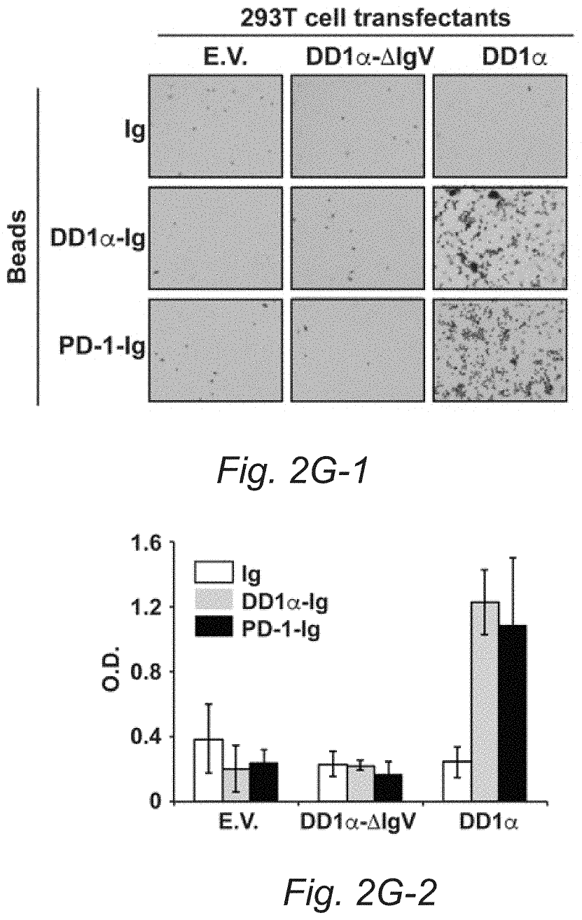

To examine whether DD1α binds other cell surface proteins involved in mediating T cell suppression, the inventors purified the membrane fraction from HCT116 human colon cancer cell lysates and performed Ig pull-down assays using DD1α-Ig and control Ig as baits, and then used mass spectrometry (MS) to identify DD1α-associated membrane proteins (FIG. 2A, Table S1). Unexpectedly, one of the abundant hits of membrane proteins identified was the co-inhibitory receptor, PD-1/PDCD1.

To validate this finding, the inventors performed Ig cell pull-down experiments using as prey transfected 293T cells expressing DD1α-HA; PD-1-HA; and another IgV family member, TIM-3-HA, that does not bind PD-1. DD1α-Ig, PD-1-Ig, PD-L1-Ig, and control Ig were used as bait (FIG. 2B). In agreement with the MS data, DD1α-Ig bound PD-1 expressing cells and DD1α expressing cells (confirming the homophilic DD1α interaction) (Yoon et al.), but not cells expressing TIM-3 (FIG. 2B). PD-1-Ig bound DD1α-HA expressing cells, but not cells expressing TIM-3 or PD-1, while PD-L1-Ig bound PD-1 transfectants but not DD1α or TIM-3 transfectants (FIG. 2B). These results indicate that DD1α specifically binds PD-1 and is a new ligand for PD-1 independent of the canonical PD-1 ligand, PD-L1.

Furthermore, the inventors performed reciprocal co-IP experiments using lysates from 293T cells transfected with either Myc-tagged DD1α plasmid, HA-tagged PD-1 plasmid, or both plasmids. Binding between DD1α-Myc and PD-1-HA occurred when either DD1α-Myc or PD-1-HA was precipitated from dual-transfected 293T cells (FIG. 2C). To assess whether DD1α and PD-1 bind directly with each other, the inventors performed Ig pull-down assays using purified His-tagged DD1α33-194 (extracellular region containing the DD1α IgV domain) with PD-1-Ig or control Ig. DD1α 33-194 bound PD-1-Ig, but not control Ig (FIG. 2D).

The inventors next performed cell-based assays to address whether the DD1α-PD-1 interaction occurs in trans across intercellular junctions. DD1α and PD-1 colocalized, as observed by immuno-electron microscopy imaging of cryosections of 293T cells transfected with both Myc-fused DD1α and HA-fused PD-1. As shown in FIG. 2E, DD1α (smaller arrowheads, labeled with 10 nm gold nano-particles) was distinctively co-localized with PD-1 (larger arrowheads, labeled with 15 nm gold nano-particles) on the cell surface.

To further confirm the specificity of intercellular binding between DD1α and PD-1, the inventors incubated DD1α-transfected Jurkat cells labeled with CF SE (Jurkat/DD1α) with PD-1 transfected 293A cells (293A/PD-1) or control 293T cells (293T), both labeled with Far Red, and measured DD1α/PD-1 intercellular binding via flow cytometry (FIG. 2F). Incubation of Jurkat/DD1α and control 293T cells produced only a small fraction of CFSE, Far Red double-positive events (2.6%), whereas incubating CFSE-labeled Jurkat/DD1α cells with Far Red-labeled 293A/PD-1 cells produced a strong increase in double-positive events (15%).

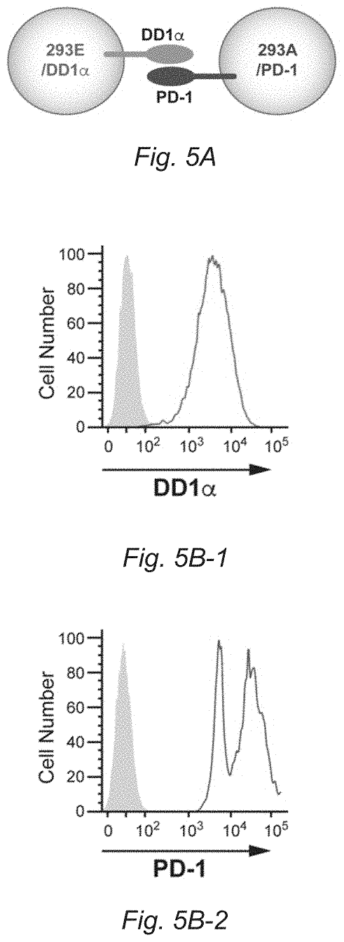

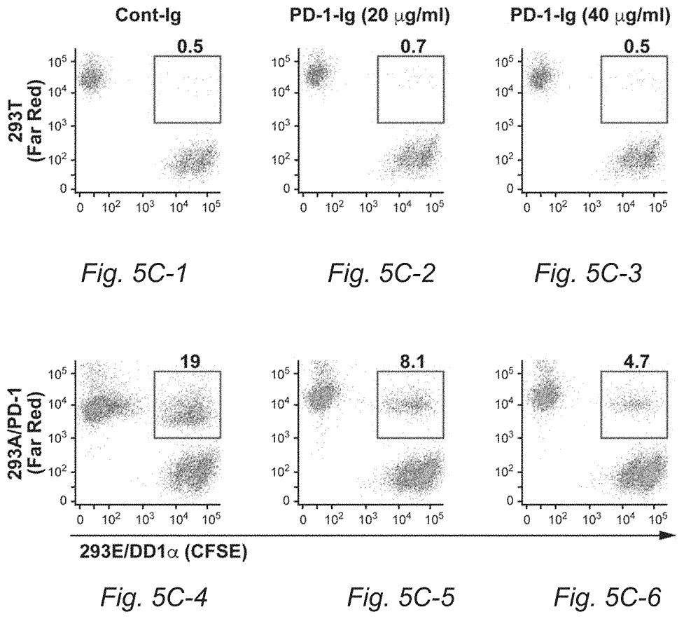

To confirm the specificity of interaction between DD1α and PD-1, the inventors tested whether soluble PD-1-Ig proteins could out-compete intercellular DD1α/PD-1 interactions, by preincubating Jurkat/DD1α cells with PD-1-Ig or control-Ig proteins prior to incubation with 293A/PD-1 cells. PD-1-Ig treatment caused a dose-dependent reduction in double positive events (FIG. 2G). To exclude the possibility that these observations may be Jurkat cell specific, the inventors further tested the intercellular binding of DD1α and PD-1 using CFSE-labeled 293E/DD1α cells and Far Red-labeled 293A/PD-1 cells, and confirmed an intercellular DD1α-PD-1 interaction in this alternative setting (FIG. 5). In summary, these cellular binding experiments indicate that DD1α and PD-1 interact via their extracellular domains and can associate between cells.

To further confirm that PD-1 interacts with the DD1α IgV domain the inventors performed bead-binding assays as previously reported (23). Beads coated with Ig-fused soluble proteins (control Ig, DD1α-Ig, or PD-1-Ig) were incubated together with transfected 293T cells expressing Wt DD1α or IgV deleted DD1α (DD1α-ΔIgV) on the cell surface. Beads coated with PD-1-Ig proteins bound to Wt DD1α on the surface of 293T cells, but deletion of the IgV domain eliminated the binding, suggesting that the DD1α-PD-1 association is mediated by the DD1α IgV domain (FIG. 2H).

### Example 3: Bidirectional Ligand/Receptor Interaction Between DD1α and PD-1 Regulates T Cell Proliferation

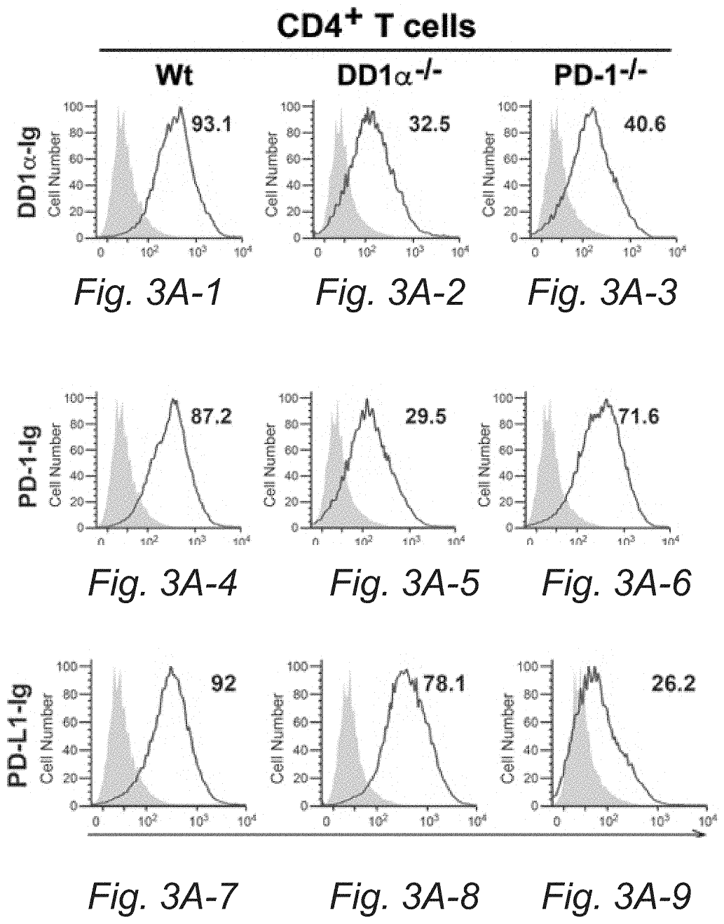

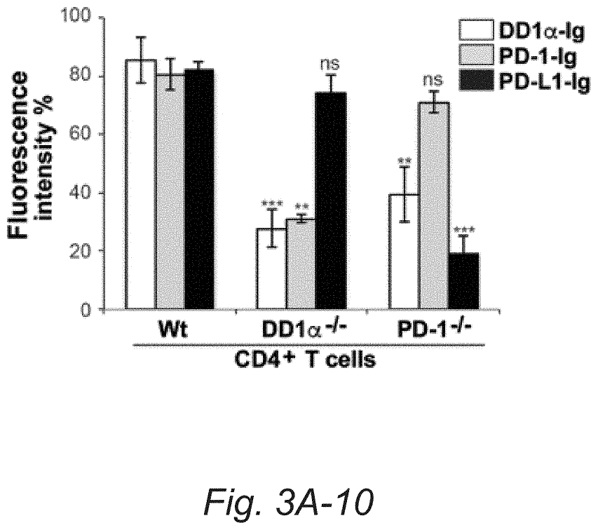

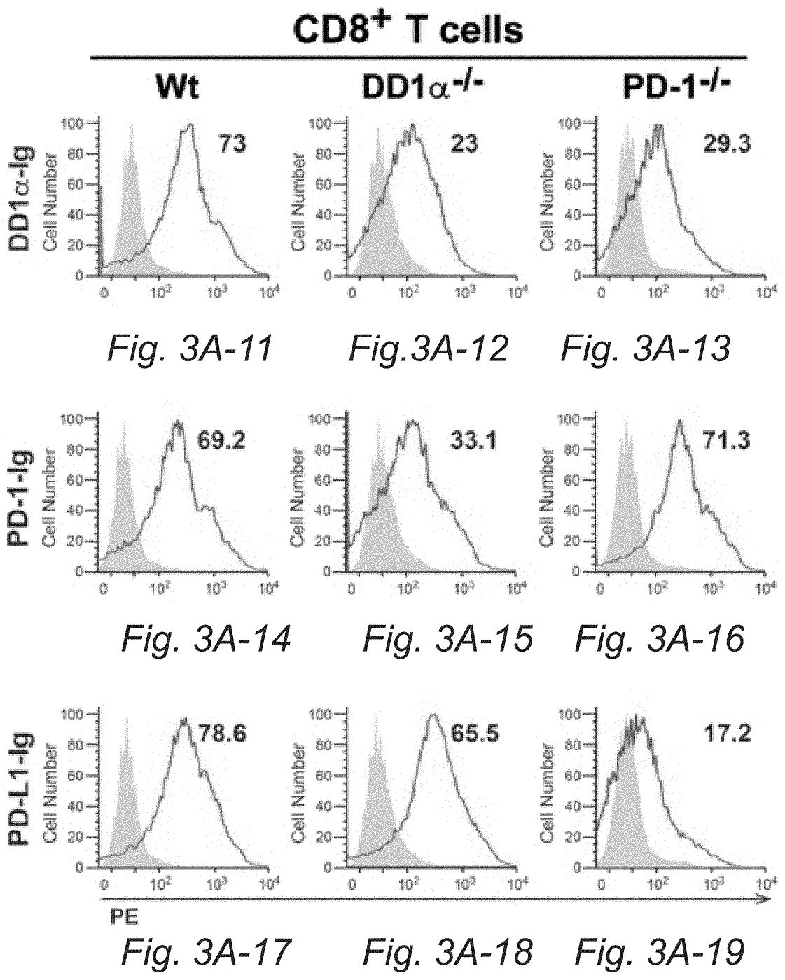

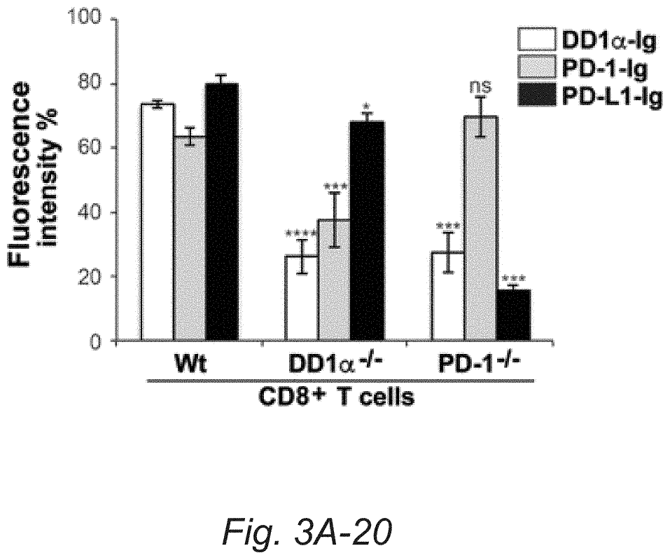

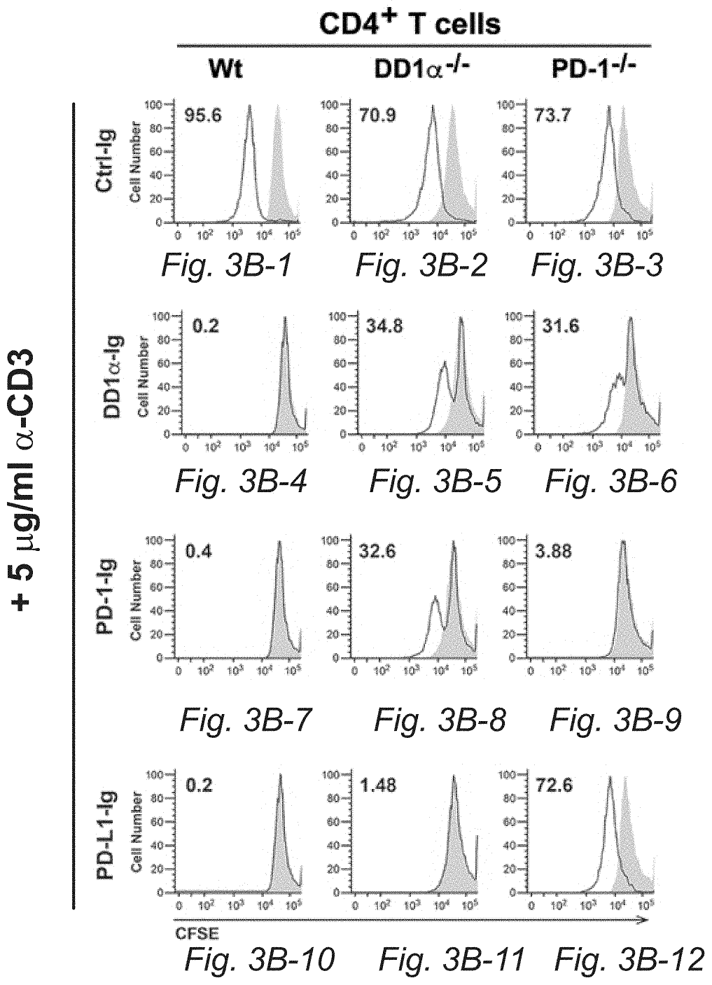

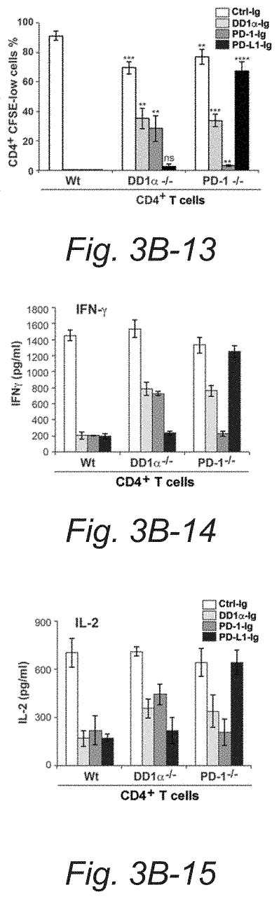

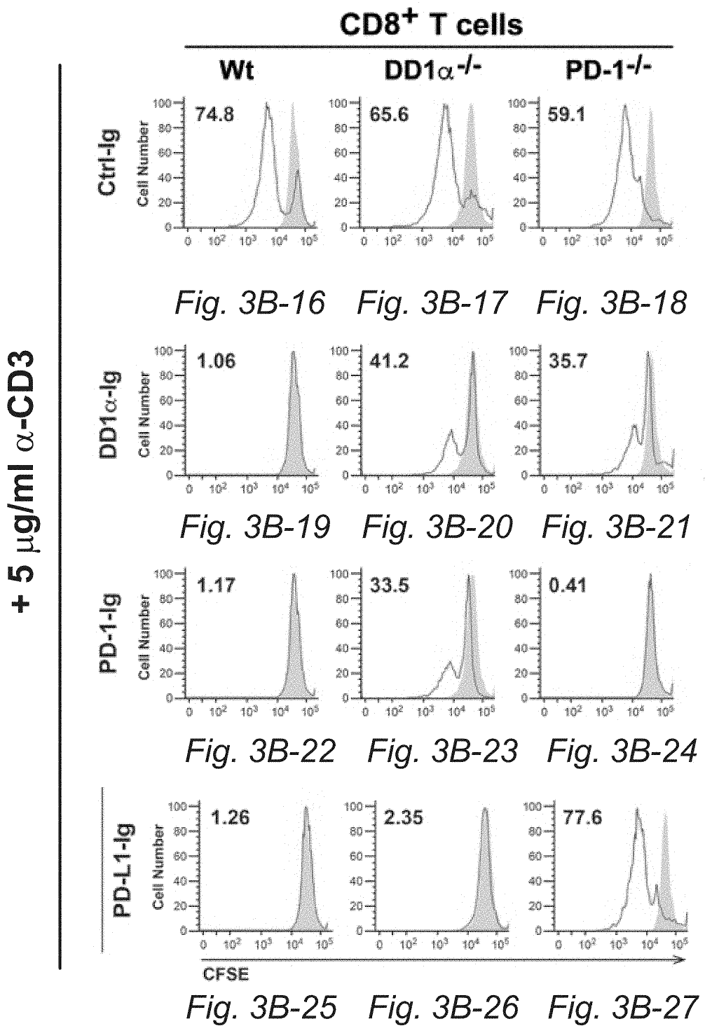

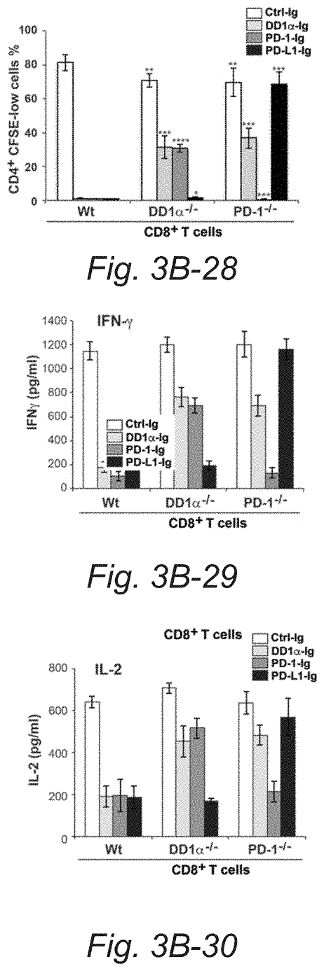

The inventors next examined the possibility that DD1α serves as a ligand for endogenous PD-1 expressed on T cells and vice versa. Using a flow cytometry approach, they first incubated soluble, fluorescently labeled DD1α-Ig fusion protein with CD4⁺ and CD8⁺ T cells isolated from Wt or DD1α-null or PD-1-null mice. DD1α-Ig bound to both CD4⁺ and CD8⁺ T cells from Wt mice, but binding of DD1α-Ig to CD4⁺ and CD8⁺ T cells from and DD1α-null and PD-1-null mice was significantly lower (FIG. 3A). Moreover, the bindings of PD-1-Ig to DD1α-null CD4⁺ or CD8⁺ T cells were significantly decreased as compared to Wt-CD4⁺ or Wt-CD8⁺ T cells (FIG. 3A).

The inventors next addressed what role DD1α signaling plays in mediating T cell suppression through PD-1. First, the inventors examined whether DD1α is able to inhibit T cell proliferation in PD-1-deficient murine CD4⁺ and CD8⁺ T cells. Treatment of Wt CD4⁺ and CD8⁺ T cells with DD1α-Ig, PD-1-Ig, or PD-L1-Ig strongly inhibited proliferation and production of cytokines IFN-γ and IL-2 induced by anti-CD3 antibody (FIG. 3B). As expected, T cell suppression by PD-L1-Ig was completely abolished in PD-1 null T cells, indicating that PD-L1 inhibits T cell activation solely through PD-1. However, T cell suppression by DD1α-Ig was partially but not completely reversed in DD1α-null and PD-1-null T cells, respectively. T cell suppression by PD-1-Ig was also reversed in DD1α-null CD4⁺ and CD8⁺ T cells but not in PD-1-null T cells. These data indicate that potential interaction between DD1α and PD-1 are important for both DD1α- and PD-1-mediated T cell inhibitory roles and that PD-1 functions as a ligand for DD1α receptor on T cells. Together, the results from binding and T cell proliferation assay experiments show that DD1α suppresses T cell activation through binding to PD-1 (FIG. 3B), suggesting that DD1α serves as a heterotypic ligand or receptor for the PD-1 checkpoint regulator that is required for its ability to mediate T cell suppression.

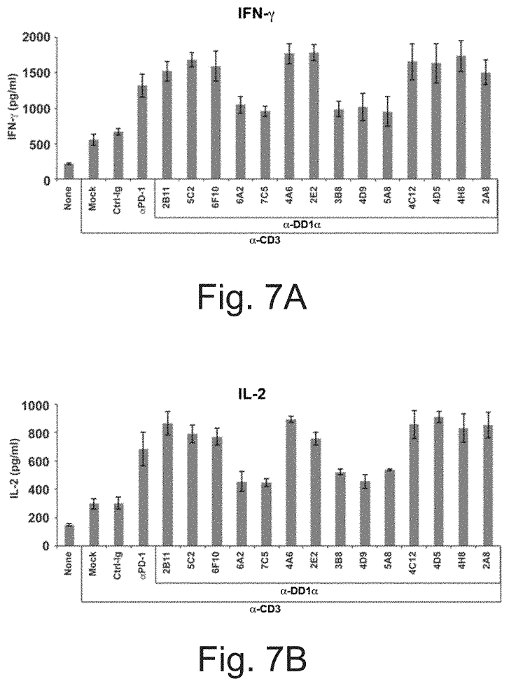

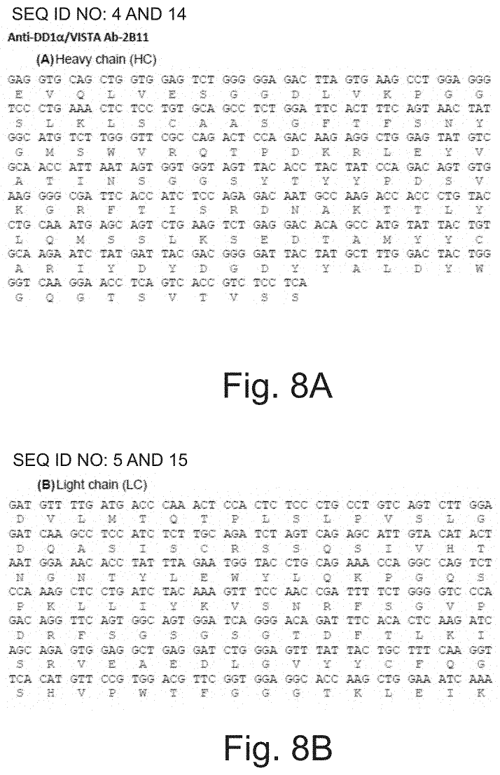

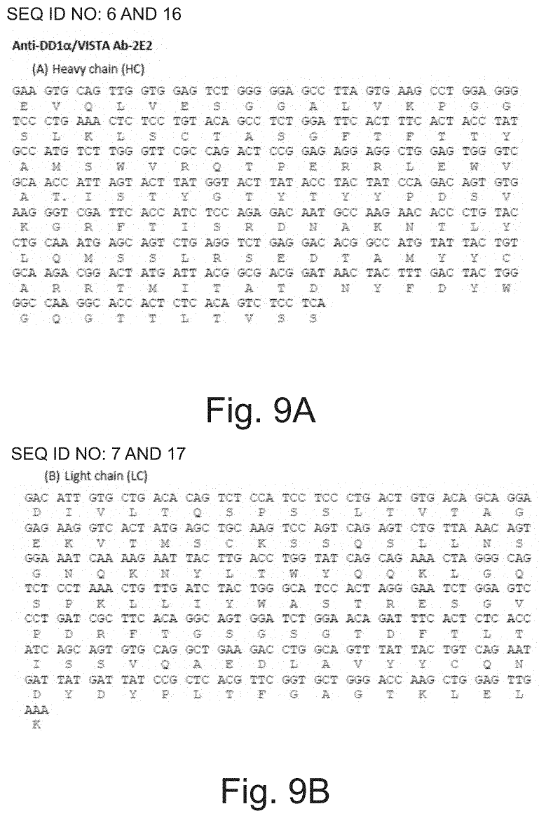

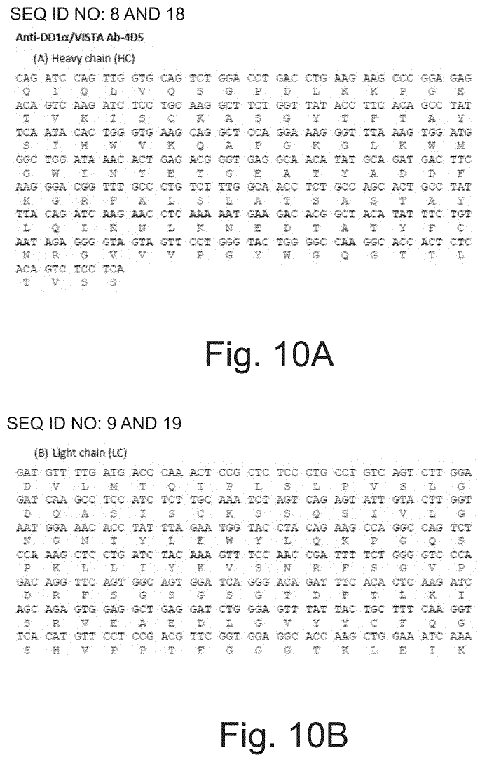

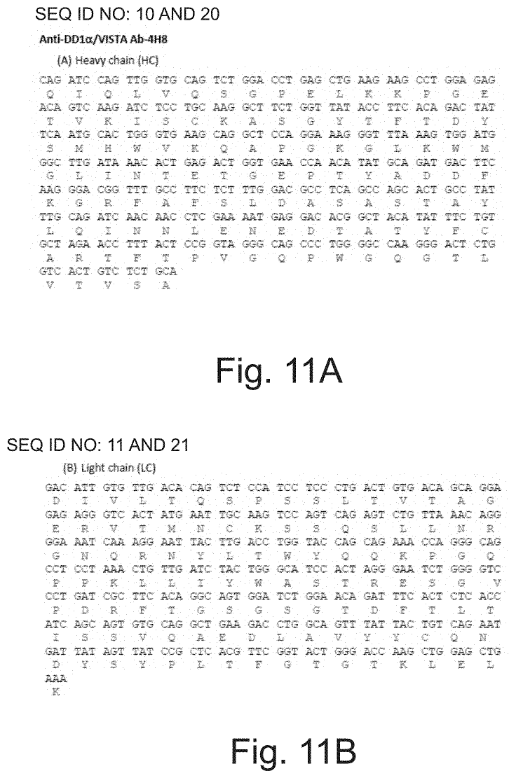

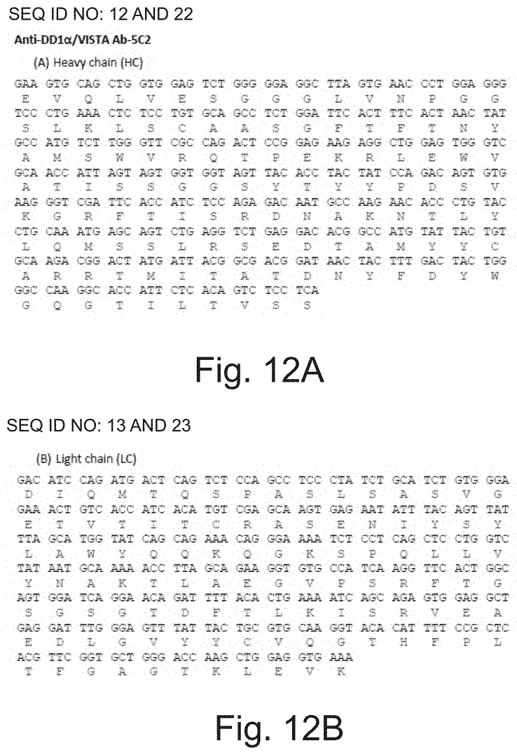

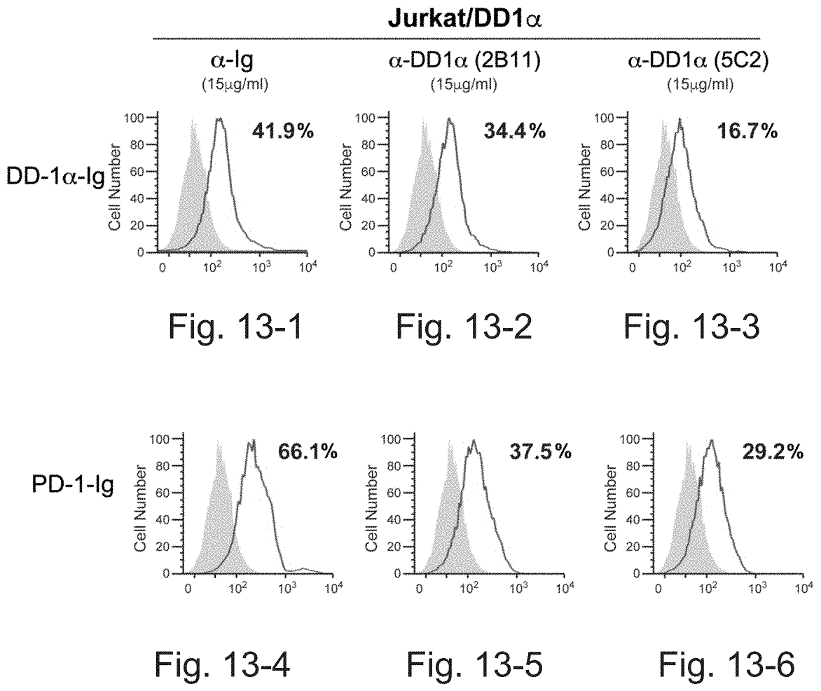

Next, the inventors generated a panel of anti-DD1α monoclonal antibodies directed against the DD1α IgV domain and screened them to identify those which prevented DD1α/PD-1 binding by competing with DD1α-Ig and PD-1-Ig for binding to Jurkat cells transfected with DD1α expression plasmid. As shown in FIG. 6, treatment with some of the DD1α-specific monoclonal Ab clones rescued induction of T cell proliferation by anti-CD3 antibody from DD1α-Ig-directed T cell suppression, and PBMC activation-induced production of IFN-γ and IL-2 was also restored (FIG. 7). Among the rescue-competent clones, we identified one (5C2) (SEQ ID NO. 12 and 13) which blocks both DD1α/DD1α and DD1α/PD-1 interactions and one (2B11) (SEQ ID NO. 4 and 5) which blocks only DD1α/PD-1 (FIG. 13). The abilities of both antibodies to restore anti-CD3 antibody-induced T cell proliferation (FIG. 6) and production of IFNγ and IL-2 (FIG. 7) confirms that both DD1α/DD1α and DD1α/PD-1 interactions mediate T cell suppression by DD1α-Ig.

### Example 4: Loss of DD1α Expression in CD11b⁺Gr-1⁺ MDSCs Enhances T Cell Proliferation

Current immune checkpoint blockade therapies are largely ineffective in patients whose tumors have a highly immunosuppressive tumor microenvironment (TME) (32-38). Tumors are often infiltrated by myeloid-derived cells such as MDSCs and tumor associated macrophages (TAMs) that suppress tumor antigen-specific T cell function and in some cases exclude T cells from tumors (37-47). High levels of MDSCs and TAMs are thus indicative of poor prognosis for cancer patients and are now targets for immunotherapy (38-45). MDSCs express immune checkpoint molecules including PD-L1, PD-L2, PD-1, and DD1α, which are thought to mediate their ability to suppress T cells. DD1α is highly expressed on MDSCs and so we sought to address what role DD1α plays in MDSC-mediated T cell suppression. MDSCs are thought to suppress T cell activity by directly binding to T cells and delivering an inhibitory signal, possibly through co-inhibitory ligands (38, 39, 42, 43).

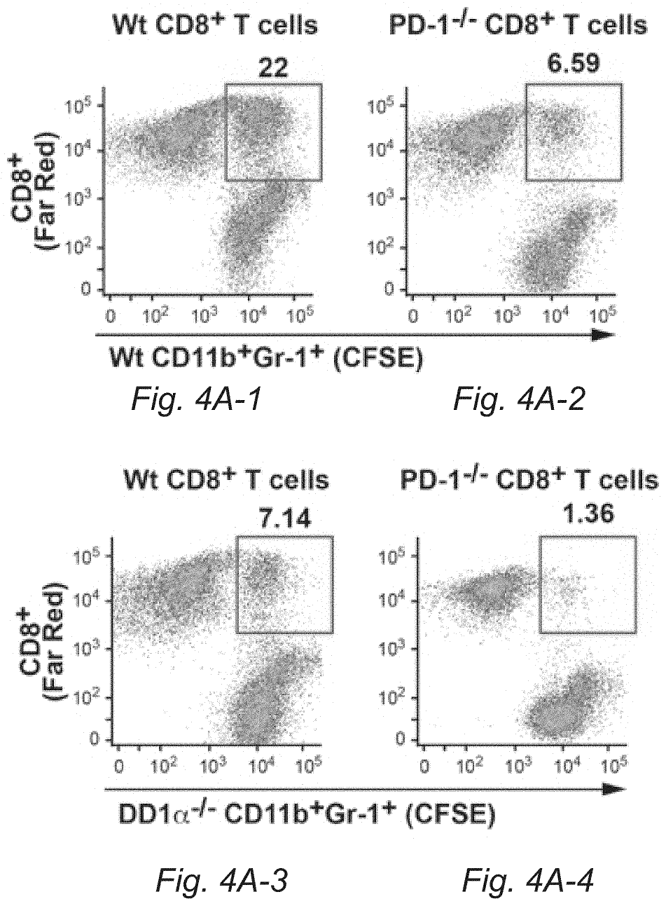

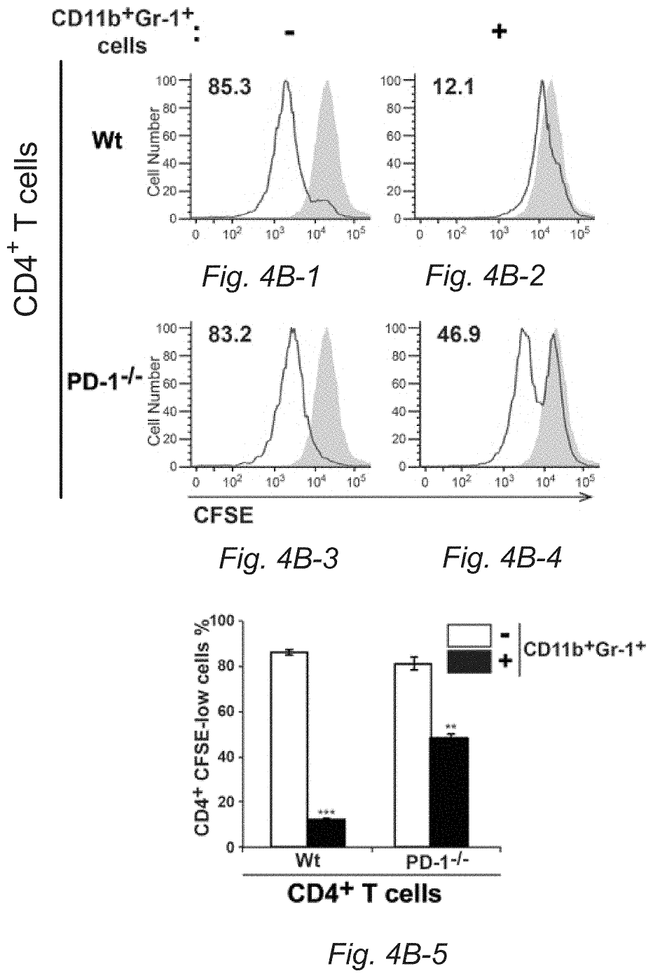

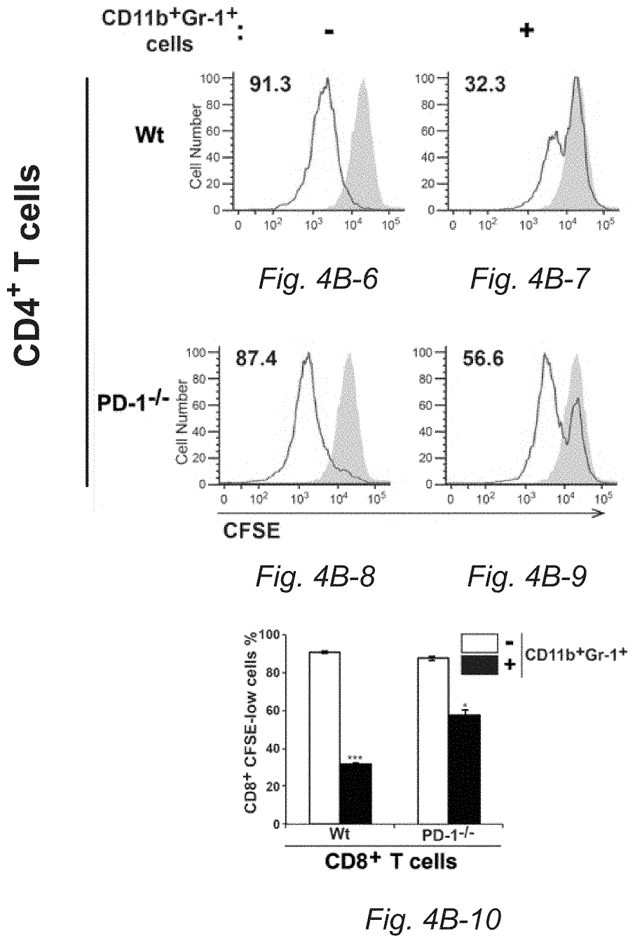

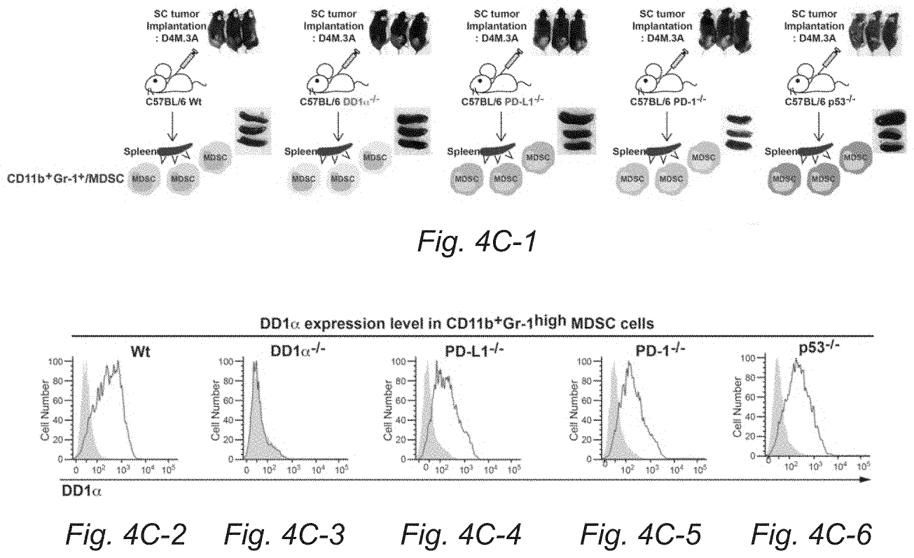

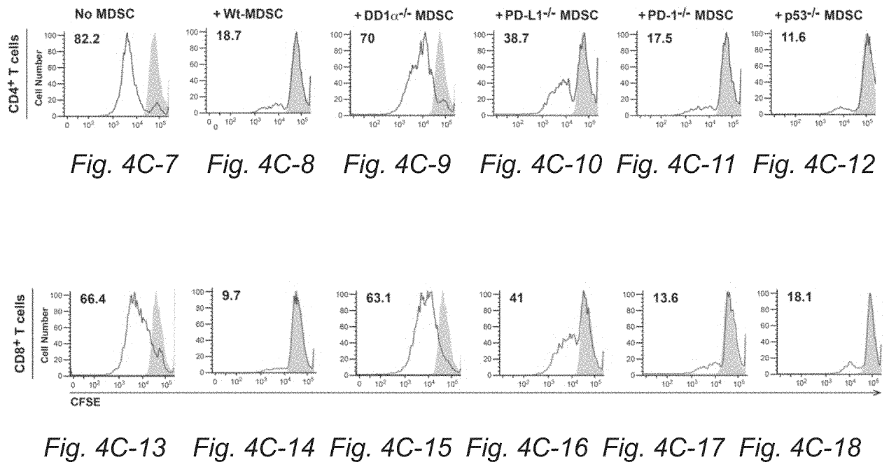

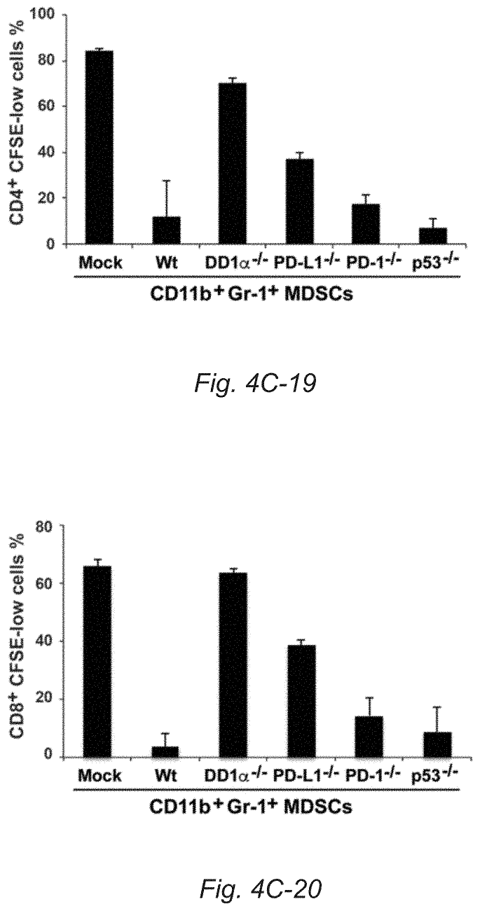

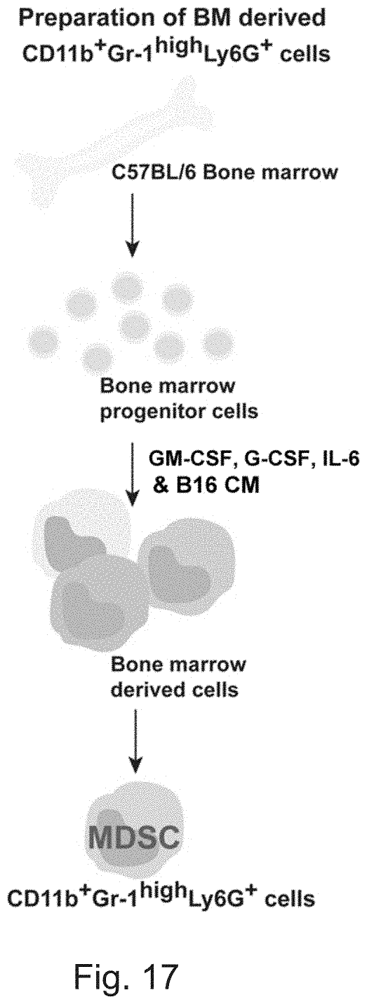

To address whether DD1α and PD-1 mediate the binding between MDSCs and T cells, the inventors isolated murine BM-derived Wt or DD1α^{-/-} CD11b⁺Gr-1⁺ MDSCs and assessed their ability to bind murine Wt or PD-1^{-/-} CD8⁺ T cells. The MDSCs were enriched with cytokines (GM-CSF, G-CSF, and IL-6) plus conditional medium of B16 mouse melanoma cells (FIG. 17). Wt and PD-1^{-/-} CD8⁺ T cells were labeled with Far Red and Wt and DD1α^{-/-} CD11b⁺Gr-1⁺ MDSCs were labeled with CFSE prior to incubating the cells together and analyzing the frequency of CFSE- and Far Red-double-positive events by flow cytometry (FIG. 4A). The inventors first compared the requirement for DD1α on CD11b⁺Gr-1⁺ MDSCs for their binding to CD8⁺ T cells. Wt CD11b⁺Gr-1⁺ MDSCs showed extensive heterotypic binding to Wt CD8⁺ T cells (22%), which was significantly reduced for DD1α-null CD11b⁺Gr-1⁺ MDSCs and Wt CD8⁺ T cells (7.1%), indicating that DD1α partially mediates the interaction between MDSCs and CD8⁺ T cells.

The inventors next assessed the requirement for PD-1 expression on CD8⁺ T cells and observed that the binding between Wt CD11b⁺Gr-1⁺ MDSCs and PD-1^{-/-} CD8⁺ T cells was also significantly decreased (6.59%) compared to Wt CD11b⁺Gr-1⁺ MDSCs and Wt CD8⁺ T cells, indicating that PD-1 also mediates the interaction between MDSCs and CD8⁺ T cells. The binding between DD1α^{-/-} CD11b⁺Gr-1⁺ MDSCs and PD-1^{-/-} CD8⁺ T cells was almost completely abolished (1.36%), indicating that DD1α on MDSCs and PD-1 on T cells are central to the interaction between MDSCs and T cells, but the interaction is likely mediated by additional DD1α and PD-1 bind partners. The inventors also addressed whether PD-1 on T cells regulates MDSC-mediated T cell suppression. The immune suppressive activity of CD11b⁺Gr-1⁺ MDSCs on CD4⁺ and CD8⁺ T cells was partially, but not completely reversed by PD-1 deficiency on the T cells (FIG. 4B), confirming that there is at least one additional receptor on T cells involved in the MDSC-directed T cell inhibition.

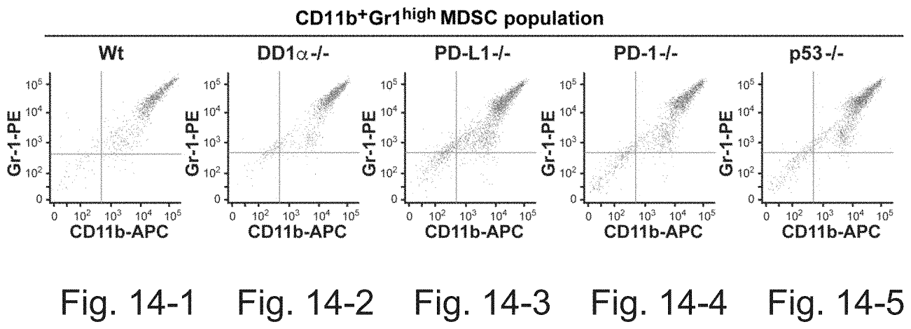

The inventors next investigated what role DD1α on MDSCs plays in MDSC-mediated suppression of T cell activity in murine tumors. First, they assessed the level of expression of DD1α on CD11b⁺Gr-1^{high} MDSCs isolated from spleens of Wt, PD-1^{-/-}, and p53^{-/-} D4M.3A tumor-bearing mice (FIG. 4C). MDSCs isolated from PD-L1^{-/-}, PD-1^{-/-} and p53^{-/-} tumor bearing mice showed similar levels of DD1α expression compared to those from Wt tumor bearing mice. In addition, no significant differences were found in the levels of the CD11b⁺Gr-1^{high} population in Wt, PD-1^{-/-}, and p53^{-/-} spleens (FIG. 14). To directly test the functional role of DD1 [symbol] in MDSC-mediated T cell suppression, the inventors incubated CD11b⁺Gr-1^{high} MDSCs isolated from spleens of tumor-bearing mice (Wt, DD1α^{-/-}, PD-1^{-/-}, and p53^{-/-}) with CFSE-labeled CD4⁺ and CD8⁺ T cells, and assessed CD3 Ab induction of T cell proliferation by flow cytometry. As anticipated, engagement of CD11b⁺Gr-1^{high} MDSCs from Wt mice strongly inhibited proliferation of CD4⁺ and CD8⁺ T cells, however, loss of DD1α on CD11b⁺Gr-1^{high} MDSCs almost completely abolished the suppression of T cell proliferation (CD4⁺ ˜70%; CD8⁺ ˜63%), to levels similar to no MDSC added (CD4 ˜82%; CD8 ˜66%) (FIG. 4C). PD-L1 deficiency also prevented CD11b⁺Gr-1^{high} MDSC-mediated suppression of T cell proliferation, but to a lower extent than loss of DD1α (FIG. 4C), indicating that PD-L1 plays only a minor role in MDSC-mediated T cell suppression compared with DD1α. Neither PD-1 deficiency nor p53 deficiency altered the MDSC inhibitory effect on T cell activity.

### Summary

Taken together, the data suggest that DD1α expression on MDSCs is critical for MDSC-mediated T cell suppression, which likely requires interaction between DD1α on MDSCs and PD-1 on T cells. The results of the aforementioned examples identify a link between post-apoptotic dead cell clearance and immune tolerance associated with a major immune checkpoint co-inhibitor PD-1 pathway. The inventors previously found that p53-dependent induction of DD1α regulates efficient removal of apoptotic cells and proper immune tolerance (23, 29). The inventors and others also demonstrated that DD1α/VISTA/PD-1H functions as an immune checkpoint regulator of T cell suppression and appears to act as both a ligand and receptor (23-25).

Proteomic and cell-based experiments additionally identified the receptor PD-1 as a novel binding partner of DD1α. Through the aforementioned examples, the inventors determined a central requirement for DD1α signaling in PD-1-mediated T cell suppression. This conclusion is supported by the observation that DD1α-mediated T cell suppression was reversed in PD-1-deficient CD4⁺ and CD8⁺ T cells, which suggests that the presence of DD1α endows PD-1 with immune co-inhibitory function. PD-1 is also expressed by macrophages and some cancer types (42, 49). It is conceivable that PD-1 may also function as a ligand for DD1α receptor on T cells. DD1α is a widely expressed membrane protein and transcriptome analysis of human tumor samples in the Cancer Genome Atlas (TCGA) showed DD1α expression in a variety of cancer types (https://portal.gdc.cancer.gov).

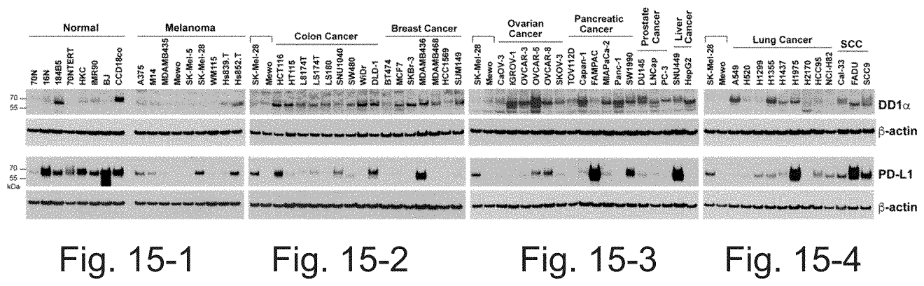

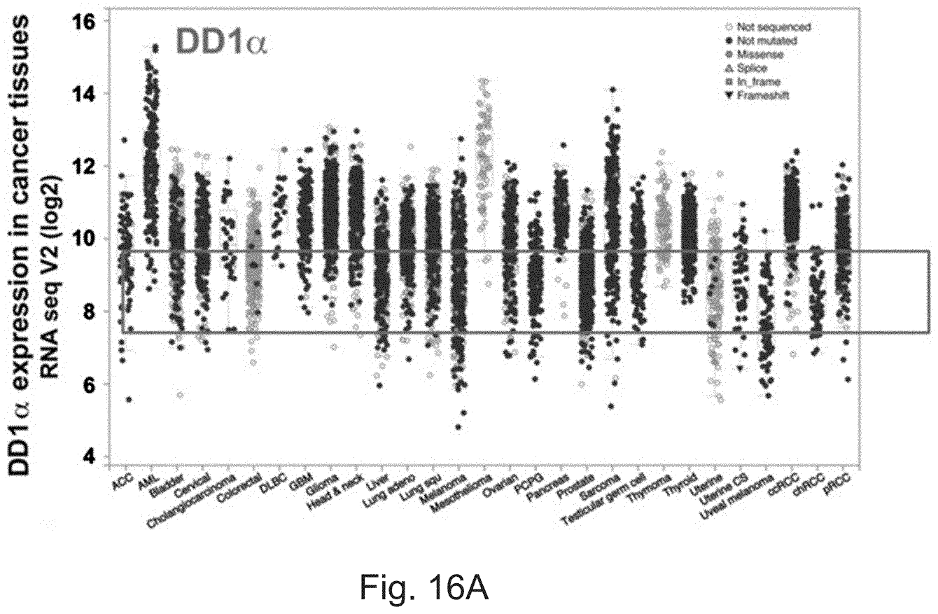

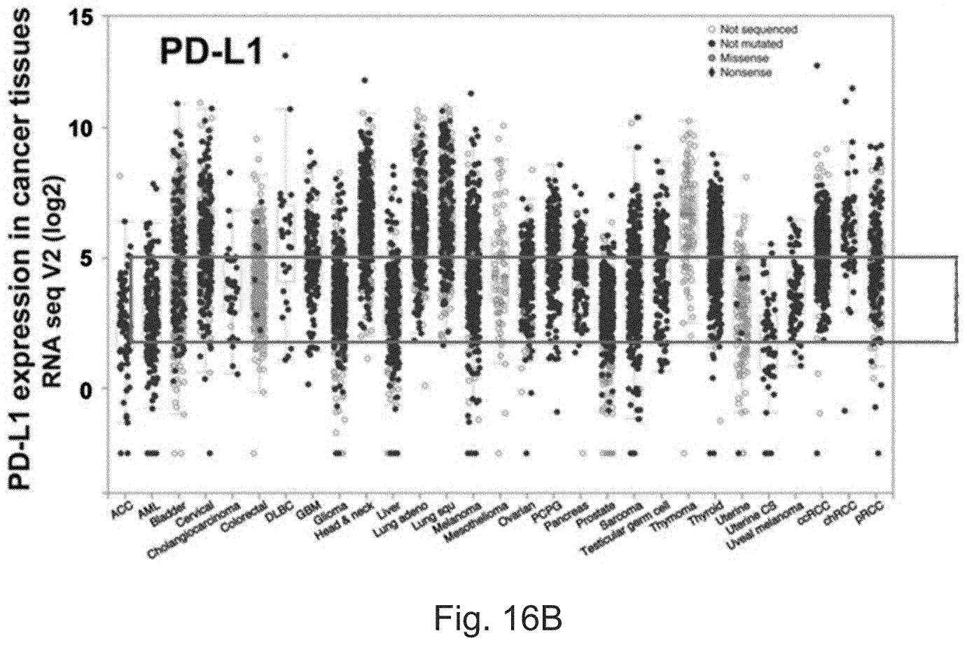

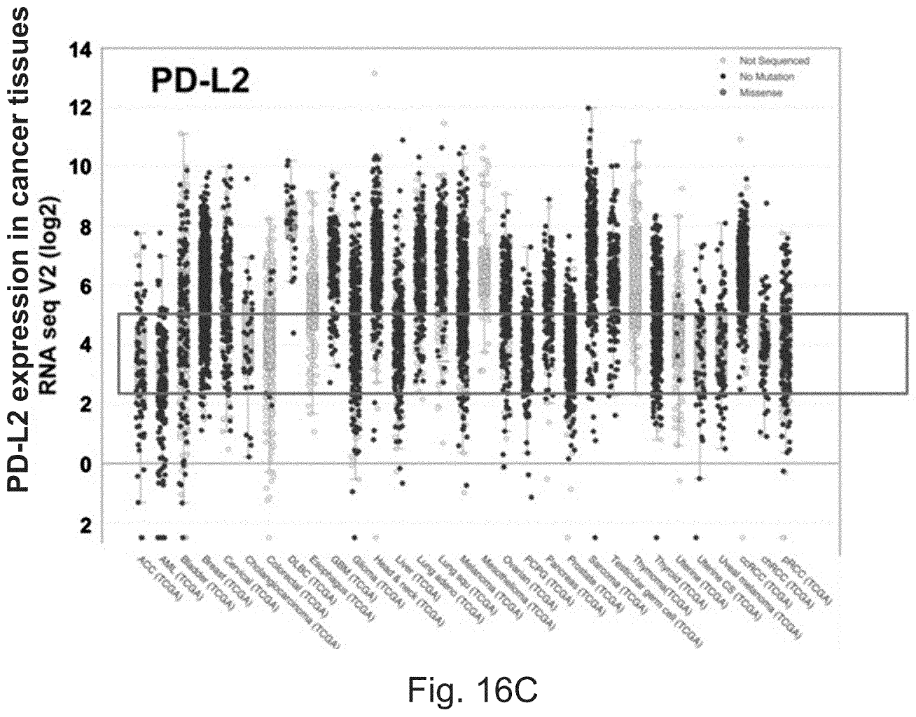

The inventors assessed expression levels of DD1α in more than 60 human cancer cell lines as compared to the levels of PD-L1, a well-known PD-1 ligand. Protein expression patterns of DD1α in these cancer cell lines are quite different from those of PD-L1 (FIG. 15), suggesting cancer-type dependent expression of these two checkpoint PD-1 ligands on cancer cells. In fact, despite remarkable progress of PD-L1/PD-1 blockade immunotherapy, acquired resistance is a major limitation of its therapeutic efficacy. It is possible that cancer cells, immunosuppressive immune cells such as MDSCs, Tregs and TAMs, and T cells may express other unidentified inhibitory receptors and ligands that compensate for the blockade of known checkpoint receptors. Importantly, during cancer therapy the mechanisms through which dying tumor cells are cleared influence tumor-specific immunity. Thus, manipulation of the immunological context of dead cell removal through the functional inhibition of DD1α during cancer therapy such as radiation and chemo-drugs leads to accumulative and enhanced immune defects that may have great potential for generation of an antitumor immunity and the control of tumor progression, and an understanding of the specific mechanisms involved warrants investigation. Targeting the DD1α/PD-1 axis may establish a new concept for managing intrinsic and acquired resistance to PD-L1/PD-1 immunotherapy in cancer. In conclusion, the observations of the aforementioned examples provide a novel link between p53-dependent DD1α-driven clearance of apoptotic cells and immune tolerance in connection with the PD-1 axis, elucidating further understanding of the mechanism(s) underlying the function of the immune checkpoint axis.

### Example 5: Exemplary Methods Utilized in the Examples Mentioned Herein

Antibodies and Reagents

The following antibodies were used for western blot analyses or flow cytometry analysis: anti-mouse DD1α (MH5A, BioLegend), anti-human PD-1 (J116, eBioscience), anti-human PD-L1 (M1H1, eBioscience), β-actin (AC-15, Sigma-Aldrich), His (Invitrogen), GST (B-14, Santa Cruz), HA (F-7, Santa Cruz), HA (C29F4, Cell Signaling Technology), Myc (9E10, Santa Cruz), anti-human CD14 APC (61D3, eBioscience), anti-mouse F4/80 APC (BM8, eBioscience), anti-mouse CD45R/B220 (RA3-6B2, eBioscience), anti-human CD4-APC (OKT4, eBioscience), anti-mouse CD4-APC (RM4-5, eBioscience), anti-human CD8-APC (SKI, eBioscience), and anti-mouse CD8a⁺-APC (53-6.7, eBioscience). Beads immobilized with anti-HA antibody (Roche) or anti-Myc (9B11, Cell signaling) were used for immunoprecipitations. Recombinant human PD-1-Ig, mouse PD-Ig, human PD-L1-Ig, mouse PD-L1-Ig, and control Ig proteins were from R&D Systems.

Cell Culture, Transfection

For DD1α-overexpressing Jurkat cells, Jurkat cells infected with lentivirus carrying DD1 [symbol] -HA were selected with 700 μg/ml geneticin, sorted, and subcloned. For PD-1 overexpressing 293A cells, 293A cells (ATCC) transfected with pcDNA3.1 plasmid carrying PD-1 were selected with hygromycin and subcloned. Apoptotic cells were induced by CPT (0.5-20 μM). To generate apoptotic thymocytes, thymocytes isolated from mice were exposed to 600 nM CPT for 12 hrs with a constant cell concentration of 1.0×10⁶ thymocytes/ml. For phagocytosis assays, apoptotic cells were labeled with pHrodo or CF SE (carboxyfluorescein diacetate succinimidyl ester) (Invitrogen). Cells were used when 60-70% of cells were apoptotic, as defined by annexin V-positive and propidium iodide-negative staining or TUNEL staining by flow cytometry. For mouse bone marrow-derived macrophages (m-BMDM), femurs and tibias were harvested from 5 to 6-week old mice and the marrow was flushed with sterile PBS using a syringe with a 26G needle. The bone marrow progenitor cell suspension was cultured in DMEM/F12 medium plus 10% FBS, 1% penicillin-streptomycin-glutamine, and 40 ng/ml recombinant murine macrophage colony stimulating factor (M-CSF, Peprotech) in non-coated bacterial culture plates. Six days later, more than 90% of the adherent cells were CD11b positive and 80% of the cells were F4/80 positive.

Purification of DD1α Monoclonal Antibodies

A DD1α mouse monoclonal Ab was raised against human DD1α (amino acids 33-311 as an immunogen). DD1α monoclonal hybridomas were cultured in Dulbecco's modified Eagle's medium (DMEM, Life Technologies, cat. no. 25030081) containing 10% FBS (Gibco), L-glutamine (Life Technologies, cat. no. 25030081), non-essential amino acids (Life Technologies, cat. no. 11140050), sodium pyruvate (Life Technologies, cat. no. 11360070), gentamicin (Life Technologies, cat. no. 15750060), and penicillin/streptomycin at 37° C. in the presence 7% CO₂. Cells were cultured up to 500 ml media at 1×10⁶/ml cell density. Monoclonal Abs were purified through protein A/G columns, eluted with 100 mM glycine (pH3), and then concentrated to 0.1˜0.5 μg/μl.

Mice and Tumor Growth In Vivo

C57BL/6 and p53^{-/-} mice were purchased from the Jackson Laboratory. DD1α^{-/-} mice were described previously (23). PD-1^{-/-} and PD-L1^{-/-} mice were provided by A. Sharpe. Mice were injected in the flank subcutaneously with 10⁶ D4M.3A melanoma cells. Tumors were measured every 3 days with a caliper. Mice were euthanized when tumors reached 10 mm. CD11b⁺Gr-1⁺ cells (MDSCs) were isolated from spleens of D4M.3A-tumor bearing Wt, DD1α^{-/-}, PD-L1^{-/-}, PD-1^{-/-}, and p53^{-/-} mice, based on the manufacture's instruction. All mice were maintained in a specific pathogen-free facility and animal experimentation was performed in accordance with the Institutional Animal Care and Use Committee Guidelines.

In Vitro Tolerance Assay

Freshly isolated thymocytes from OVA transgenic mice were treated with 600 nM CPT for 12 hours. Untreated thymocytes were used as a negative control. Live or apoptotic thymocytes were incubated with Wt macrophages (mBMDMs) for 3 hours, then un-engulfed cells were removed by washing with PBS. Phagocytic macrophages were pre-incubated with MH5A mAb (20 μg/ml) (BioLegend) or isotype IgG1 control Ab for 30 min prior to co-culture with OT-II CD4⁺ T cells for 72 hours. In addition, phagocytic macrophages were co-cultured with OT-II CD4⁺ T cells from OT-II transgenic mice, which had been pre-incubated with MH5A mAb or isotype IgG1 control Ab for 3 hours. 72 hours later, proliferating T cells were labeled with CFSE (1 μM) and the percentage of proliferating cells was determined by flow cytometry. Data represent means±SD (n=3). **P<0.0005, *P<0.005 (Tukey's t test).

T Cell Tolerance Animal Models

For OVA antigen-specific T-cell tolerance induction, OVA peptide was used as antigen according to previous studies (30, 31). Briefly, the transgenic OT-IIRag2^{-/-} OT-II DD1α^{-/-}Rag2^{-/-}, or OTII PD-1^{-/-}Rag2^{-/-} mice were injected intraperitoneally with 500 μg of OVA peptide (323-339, 323-ISQAVHAAHAEINEAGR-339) in an equal volume amount of complete Freund's adjuvant (CFA) or PBS alone at day 0 (stimulation stage for immune response) and at day 4 (tolerance induction stage). At day 10, CD4⁺ T cells were isolated from the spleen and pooled peripheral lymphocytes from each experimentally treated mouse. Then, the isolated CD4⁺ T cells from each mouse were re-stimulated in vitro with various concentrations of OVA₃₂₃₋₃₃₉ peptide (0.1, 1, 2.5, and 10 μg/ml) in 96-well plates. The 96-well cultures were set up with a total cell number of 2.5×10⁴ in 200 μl RPMI complete medium containing EdU from a Click-iT™ EdU Alexa Fluor™ 488 Imaging Kit (Thermo Fisher Scientific). After incubation for 72 hour, EdU-labeled cells were treated according to the manufacturer's protocol (ThermoFisher Scientific) and then analyzed by flow cytometry. Pull down assay and Mass spectrometry HCT116 human colon carcinoma cells (ATCC) were cultured on 150 mm culture dishes (40 plates) until 80% confluent. Cells were washed twice with ice-cold PBS, harvested, and resuspended in 1 ml of subcellular fractionation (SF) buffer (250 mM sucrose, 20 mM HEPES, pH7.4, 10 mM KCl, 1.5 mM MgCl₂, 1 mM EDTA, 1 mM EGTA, 1 mM DTT) per 150 mm plate. Cells were pelleted at 720×g at 4° C. for 10 min. Membrane fractions were separated through ultracentrifugation at 100,000×g at 4° C. for 1 hour. Membrane fractionated proteins were used for screening of binding partners. For pull down assay against Ig fusion DD1α protein, DD1α-Ig protein was immobilized on protein A/G agarose and incubated with the membrane fractions. The bound proteins were separated through SDS-PAGE, stained with coomassie blue dye, and then stained gel slices (˜25 to 75 kD) were excised for mass spectrometry.

Immunoelectron Microscopy

After the incubation of DD1α-myc-transfected 293T cells and PD-1-HA-transfected U2OS cells, cell pellets were fixed with 4% PFA+0.1% glutaraldehyde in PBS. Immuno-gold electron microscopy using double immunogold labeling with anti-myc (10 nm gold) and anti-HA (15 nm gold) was performed in the Harvard Medical School Electron Microscopy Facility using Tokayasu methods.

Intercellular Interaction Assay

Jurkat/DD1α, 293E/DD1α, and CD11b⁺Gr-1^{high}Ly6G⁺ cells were stained by 504 carboxyfluorescein succinimidyl ester (CFSE, Life Technologies, C34554) for 5 min, and 293A/PD-1, 293T, 293A/PD-1, 300.19, and 300.19/PD-1 cells were stained by 1 μM Far Red (Life Technologies, C34564) for 5 min. The stained cells were washed twice with binding buffer (0.5% BSA, 1 mM CaCl₂), 1 mM MgCl₂ in PBS). Two differently stained cells were incubated in one tube for 1 hour, and then analyzed by flow cytometry.

T Cell-Ig Fusion Protein Binding Assay

CD4⁺ T cells were isolated using CD4⁺ T cell isolation kits (Miltenyl Biotec, 130-104-454 (for mouse) and 130-096-533 (for human)). CD8⁺ T cells were isolated using CD8⁺ T cell isolation kits (Miltenyl Biotec, 130-104-075 (for mouse) and 130-096-495 (for human)). 2×10⁵ isolated T cells were seeded per well in a 96 well plate coated with 5 μg/ml α-CD3 (eBioscience, 16-0031-85 (for mouse), 16-0039-85 (for human)). After 72 hour, α-CD3-activated T cells were incubated with 40 μg/ml Ig fusion protein for 1 hour. Cells were then washed once with PBS and incubated with anti-human PE-conjugated secondary Ab (Southern Biotech, 2043-09) for 30 min, and then the cells were analyzed by flow cytometry.

In Vitro T Cell Activation and Suppression Assay

CD4⁺ and CD8⁺ T cells were isolated from C57BL/6 mouse spleens using a Miltenyl CD4+ or CD8+ T cell Isolation Kit and labeled with the cell proliferation tracer CFSE following the protocols provided by the manufacturer. One million CFSE-labeled T cells per well were cultured in 5 μg/ml α-CD3-coated 96-well plates with 200 μl/well of complete RPMI-1640 media. After 72 hours, cells were harvested and analyzed by flow cytometry.

Binding Assay with Blue Latex Beads Conjugated Proteins

DD1α-Ig (R&D Systems, 7126-B7), PD-1-Ig (Sino Biological, 10377-H03H), and Ig protein (Sino Biological, 10702-HNAH) were covalently coupled to carboxyl microparticles by EDAC solution (PolyLink, Polysciences, Inc., 24350-1) following the manufacturer's protocol. Full length DD1α-, DD1α-ΔIgV-, or empty vector-transfected 293T cells were incubated with the Ig protein conjugated beads in PBS containing 2% FBS at room temperature for 30 min. Cells interacting with beads were investigated by inverted microscope and the absorbance at 280 nm was measured.

Phagocytosis Assay

To differentiate bone marrow-derived macrophages (BMDM), bone marrow progenitor cells were obtained from mouse femurs and tibias and cultured for 6 days as described above in Cell culture, transfection. After 6 days, adherent cells were used for phagocytosis assay. For flow cytometry analysis of phagocytosis, 2.0×105 BMDM were plated in 24 well non-treated plates (Fisher scientific) 1 day before the phagocytosis assay. To induce apoptosis in thymocytes, cells were treated with 1 μM CPT for 6 hours. Apoptotic thymocytes were stained with 20 pg/ml pHrodo (Invitrogen) for 30 min at RT. BMDM were starved for 30 min in DMEM/F12 containing 2% FBS and then incubated with 4×106 pHrodo-stained apoptotic thymocytes for 30 min. The cells were then analyzed by flow cytometry.

Cell Isolation and Magnetic Cell Sorting

Human peripheral blood mononuclear cells (PBMCs) were isolated from buffy coats from normal blood obtained from the Massachusetts General Hospital Blood Donor Center (protocol number 2012P002174). CD4⁺ T cells and CD8⁺ T cells were purified from PBMC using CD4 microbeads and CD8 microbeads (Miltenyi) and cultured in RPMI-1640 supplemented with 10% FBS (Gibco), β-mercaptoethanol, L-glutamine, and antibiotics. The T cells were stimulated with plate-bound anti-CD3 (OKT3).

Mouse BM Cultures and MDSC Generation

To obtain bone marrow-derived CD11b⁺Gr-1⁺ cells, bone marrow progenitor cells were obtained from mouse femurs and tibias as described above in Cell culture, transfection and 2.5×10⁶ cells were seeded into 100 mm dishes in 10 ml of medium supplemented with different combinations of GM-CSF (20 ng/ml), G-CSF (20 ng/ml), IL-6 (20 ng/ml), and 10% conditioned medium of D4M.3A melanoma cells. After 4 days, bone marrow-derived CD11b⁺Gr-1⁺ cells were isolated by Myeloid-Derived Suppressor Cell (MDSC) Isolation Kit (Miltenyi Biotech, 130-094-538). Purity was confirmed to be over 90% by flow cytometry.

Generation of MDSC from Tumor Bearing Mice

To prepare tumor-bearing mice, 1×10⁶ D4M.3A melanoma cells were injected subcutaneously into the right flank of C57BL/6 mice. Spleens were excised from tumor-bearing mice and CD11b⁺Gr-1⁺ cells were isolated by MDSC isolation kit (Miltenyi Biotech).

Statistical Analysis

Student's two-tailed t-test was used to calculate statistical significance for difference in a particular measurement between two groups. P values of <0.05 were considered statistically significant.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

S. Arandjelovic, K. S. Ravichandran, Phagocytosis of apoptotic cells in homeostasis. Nat Immunol 16, 907-917 (2015).
M. R. Elliott, K. S. Ravichandran, The Dynamics of Apoptotic Cell Clearance. Dev Cell 38, 147-160 (2016).
T. A. Ferguson, J. Choi, D. R. Green, Armed response: how dying cells influence T-cell functions. Immunol Rev 241, 77-88 (2011).
Y. Fuchs, H. Steller, Programmed cell death in animal development and disease. Cell 147, 742-758 (2011).
T. S. Griffith, T. A. Ferguson, Cell death in the maintenance and abrogation of tolerance: the five Ws of dying cells. Immunity 35, 456-466 (2011).
S. Nagata, R. Hanayama, K. Kawane, Autoimmunity and the clearance of dead cells. Cell 140, 619-630 (2010).
S. Nagata, M. Tanaka, Programmed cell death and the immune system. Nat Rev Immunol 17, 333-340 (2017).
S. Nagata, Apoptosis and autoimmune diseases. Ann N Y Acad Sci 1209, 10-16 (2010).
A. M. Fond, K. S. Ravichandran, Clearance of Dying Cells by Phagocytes: Mechanisms and Implications for Disease Pathogenesis. Adv Exp Med Biol 930, 25-49 (2016).
I. K. Poon, C. D. Lucas, A. G. Rossi, K. S. Ravichandran, Apoptotic cell clearance: basic biology and therapeutic potential. Nat Rev Immunol 14, 166-180 (2014).
L. Galluzzi, A. Buque, O. Kepp, L. Zitvogel, G. Kroemer, Immunogenic cell death in cancer and infectious disease. Nat Rev Immunol 17, 97-111 (2017).
P. Sharma, J. P. Allison, The future of immune checkpoint therapy. Science 348, 56-61 (2015).
C. Munoz-Fontela, A. Mandinova, S. A. Aaronson, S. W. Lee, Emerging roles of p53 and other tumour-suppressor genes in immune regulation. Nat Rev Immunol 16, 741-750 (2016).
P. Sharma, J. P. Allison, Immune checkpoint targeting in cancer therapy: toward combination strategies with curative potential. Cell 161, 205-214 (2015).
D. M. Pardoll, The blockade of immune checkpoints in cancer immunotherapy. Nat Rev Cancer 12, 252-264 (2012).
S. H. Baumeister, G. J. Freeman, G. Dranoff, A. H. Sharpe, Coinhibitory Pathways in Immunotherapy for Cancer. Annu Rev Immunol 34, 539-573 (2016).
V. A. Boussiotis, Molecular and Biochemical Aspects of the PD-1 Checkpoint Pathway. N Engl J Med 375, 1767-1778 (2016).
A. H. Sharpe, Introduction to checkpoint inhibitors and cancer immunotherapy. Immunol Rev 276, 5-8 (2017).
S. C. Casey et al., MYC regulates the antitumor immune response through CD47 and PD-L1. Science 352, 227-231 (2016).
W. Zou, J. D. Wolchok, L. Chen, PD-L1 (B7-H1) and PD-1 pathway blockade for cancer therapy: Mechanisms, response biomarkers, and combinations. Sci Transl Med 8, 328rv324 (2016).
J. M. Pitt et al., Resistance Mechanisms to Immune-Checkpoint Blockade in Cancer: Tumor-Intrinsic and -Extrinsic Factors. Immunity 44, 1255-1269 (2016).
A. H. Sharpe, K. E. Pauken, The diverse functions of the PD1 inhibitory pathway. Nat Rev Immunol 18, 153-167 (2018).
K. W. Yoon et al., Control of signaling-mediated clearance of apoptotic cells by the tumor suppressor p53. Science 349, 1261669 (2015).
D. B. Flies et al., Coinhibitory receptor PD-1H preferentially suppresses CD4(+) T cell-mediated immunity. J Clin Invest 124, 1966-1975 (2014).
L. Wang et al., VISTA, a novel mouse Ig superfamily ligand that negatively regulates T cell responses. J Exp Med 208, 577-592 (2011).
J. L. Lines et al., VISTA is an immune checkpoint molecule for human T cells. Cancer Res 74, 1924-1932 (2014).
I. Le Mercier et al., VISTA Regulates the Development of Protective Antitumor Immunity. Cancer Res 74, 1933-1944 (2014).
J. Gao et al., VISTA is an inhibitory immune checkpoint that is increased after ipilimumab therapy in patients with prostate cancer. Nat Med 23, 551-555 (2017).
L. Zitvogel, G. Kroemer, CANCER. A p53-regulated immune checkpoint relevant to cancer. Science 349, 476-477 (2015).
Y. H. Huang et al., CEACAM1 regulates TIM-3-mediated tolerance and exhaustion. Nature 517, 386-390 (2015).
E. R. Kearney, K. A. Pape, D. Y. Loh, M. K. Jenkins, Visualization of peptide-specific T cell immunity and peripheral tolerance induction in vivo. Immunity 1, 327-339 (1994).
D. S. Chen, I. Mellman, Oncology meets immunology: the cancer-immunity cycle. Immunity 39, 1-10 (2013).
K. G. Anderson, I. M. Stromnes, P. D. Greenberg, Obstacles Posed by the Tumor Microenvironment to T cell Activity: A Case for Synergistic Therapies. Cancer Cell 31, 311-325 (2017).
J. M. Pitt et al., Targeting the tumor microenvironment: removing obstruction to anticancer immune responses and immunotherapy. Ann Oncol 27, 1482-1492 (2016).
M. D. Vesely, M. H. Kershaw, R. D. Schreiber, M. J. Smyth, Natural innate and adaptive immunity to cancer. Annu Rev Immunol 29, 235-271 (2011).
T. F. Gajewski, H. Schreiber, Y. X. Fu, Innate and adaptive immune cells in the tumor microenvironment. Nat Immunol 14, 1014-1022 (2013).
D. Marvel, D. I. Gabrilovich, Myeloid-derived suppressor cells in the tumor microenvironment: expect the unexpected. J Clin Invest 125, 3356-3364 (2015).
V. Kumar, S. Patel, E. Tcyganov, D. I. Gabrilovich, The Nature of Myeloid-Derived Suppressor Cells in the Tumor Microenvironment. Trends Immunol 37, 208-220 (2016).
D. I. Gabrilovich, Myeloid-Derived Suppressor Cells. Cancer Immunol Res 5, 3-8 (2017).
A. Mantovani, F. Marchesi, A. Malesci, L. Laghi, P. Allavena, Tumour-associated macrophages as treatment targets in oncology. Nat Rev Clin Oncol 14, 399-416 (2017).
R. Noy, J. W. Pollard, Tumor-associated macrophages: from mechanisms to therapy. Immunity 41, 49-61 (2014).
S. R. Gordon et al., PD-1 expression by tumour-associated macrophages inhibits phagocytosis and tumour immunity. Nature 545, 495-499 (2017).
R. A. Franklin et al., The cellular and molecular origin of tumor-associated macrophages. Science 344, 921-925 (2014).
J. M. Brown, L. Recht, S. Strober, The Promise of Targeting Macrophages in Cancer Therapy. Clin Cancer Res 23, 3241-3250 (2017).
A. Mantovani, A. Sica, Macrophages, innate immunity and cancer: balance, tolerance, and diversity. Curr Opin Immunol 22, 231-237 (2010).
J. W. Pollard, Tumour-educated macrophages promote tumour progression and metastasis. Nat Rev Cancer 4, 71-78 (2004).
B. Z. Qian, J. W. Pollard, Macrophage diversity enhances tumor progression and metastasis. Cell 141, 39-51 (2010).
S. Kleffel et al., Melanoma Cell-Intrinsic PD-1 Receptor Functions Promote Tumor Growth. Cell 162, 1242-1256 (2015).

What is claimed is:

1. A composition for treating immune-related diseases including cancer, comprising
at least one isolated monoclonal antibody that modulates DD1α activity by binding to a DD1α polypeptide consisting of SEQ ID NO:1, wherein the binding of the at least one isolated monoclonal antibody to the DD1α polypeptide disrupts the physical association between the DD1α polypeptide on the surface of a first cell and a separate polypeptide on the surface of a second cell; and
a pharmaceutically acceptable carrier or diluent.

2. The composition of claim 1, further comprising an anti-cancer therapeutic.

3. The composition of claim 1, wherein the polypeptide on the surface of the second cell is a DD1α polypeptide.

4. The composition of claim 1, wherein the polypeptide on the surface of the second cell is a PD-1 polypeptide.

5. The composition of claim 3, wherein the first cell is a tumor-associated myeloid cell, and wherein the second cell is a T cell.

6. The composition of claim 4, wherein the first cell is a tumor-associated myeloid cell, and wherein the second cell is a T cell.

7. The composition of claim 5, wherein the at least one isolated monoclonal antibody is chosen from the group of antibodies having a heavy chain amino acid sequence at least 95% identical to SEQ ID NO. 4 and a light chain amino acid sequence at least 95% identical to SEQ ID NO. 5, a heavy chain amino acid sequence at least 95% identical to SEQ ID NO. 6 and a light chain amino acid sequence at least 95% identical to SEQ ID NO. 7, a heavy chain amino acid sequence at least 95% identical to SEQ ID NO. 8 and a light chain amino acid sequence at least 95% identical to SEQ ID NO. 9, a heavy chain amino acid sequence at least 95% identical to SEQ ID NO. 10 and a light chain amino acid sequence at least 95% identical to SEQ ID NO. 11, a heavy chain amino acid sequence at least 95% identical to SEQ ID NO. 12 and a light chain amino acid sequence at least 95% identical to SEQ ID NO.13, and combinations thereof.

8. The composition of claim 6, wherein the at least one isolated monoclonal antibody is chosen from the group of antibodies having a heavy chain amino acid sequence at least 95% identical to SEQ ID NO. 4 and a light chain amino acid sequence at least 95% identical to SEQ ID NO. 5, a heavy chain amino acid sequence at least 95% identical to SEQ ID NO. 6 and a light chain amino acid sequence at least 95% identical to SEQ ID NO. 7, a heavy chain amino acid sequence at least 95% identical to SEQ ID NO. 8 and a light chain amino acid sequence at least 95% identical to SEQ ID NO. 9, a heavy chain amino acid sequence at least 95% identical to SEQ ID NO. 10 and a light chain amino acid sequence at least 95% identical to SEQ ID NO. 11, a heavy chain amino acid sequence at least 95% identical to SEQ ID NO. 12 and a light chain amino acid sequence at least 95% identical to SEQ ID NO.13, and combinations thereof.

9. A method for providing immunotherapy to a subject having cancer, comprising the steps of:
identifying a subject with a cancer that is likely to respond to the immunotherapy, wherein the likelihood of response is determined by
a. obtaining a bodily fluid or tissue sample from a subject diagnosed with cancer,
b. measuring the fraction of tumor cells in the sample expressing DD1α on the surface of the cell,
c. comparing the fraction to a reference sample, and
d. determining whether the fraction of tumor cells has surpassed a determined threshold with respect to the reference sample; and
administering to the subject a composition comprising at least one isolated monoclonal antibody and a pharmaceutically acceptable carrier or diluent, wherein the at least one isolated monoclonal antibody in the composition binds to a DD1α polypeptide consisting of SEQ ID NO:1, and wherein the binding of the at least one isolated monoclonal antibody to the DD1α polypeptide disrupts the physical association between the DD1α polypeptide on the surface of a first cell and a separate polypeptide on the surface of a second cell.

10. The method of claim 9, wherein the polypeptide on the surface of the second cell is a DD1α polypeptide.

11. The method of claim 9, wherein the polypeptide on the surface of the second cell is a PD-1 polypeptide.

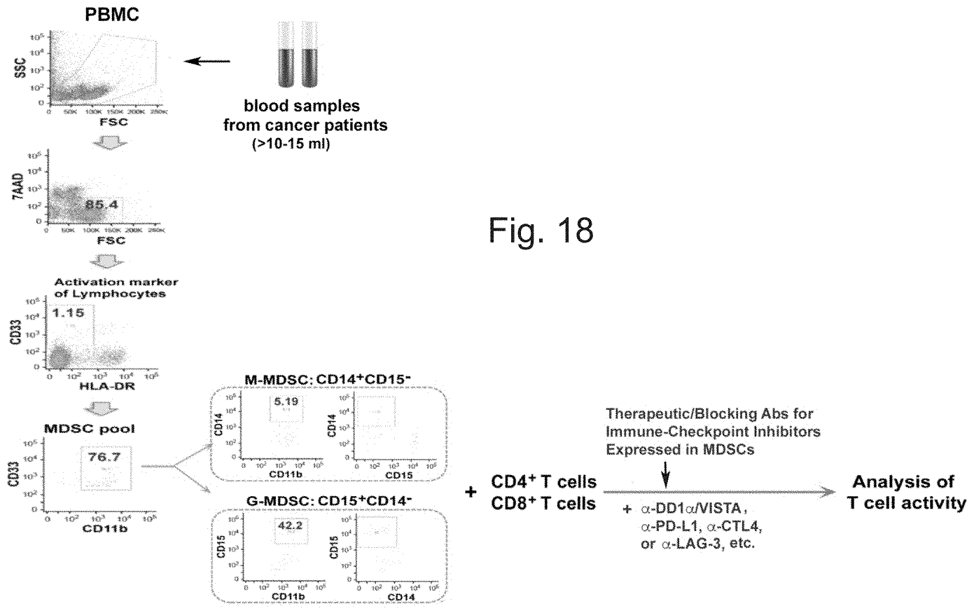

12. The method of claim 10, wherein the first cell is a tumor-associated myeloid cell, and wherein the second cell is a T cell (FIG. 18).

13. The method of claim 11, wherein the first cell is a tumor-associated myeloid cell, and wherein the second cell is a T cell (FIG. 18).

14. The method of claim 12, wherein the at least one isolated monoclonal antibody is chosen from the group of antibodies having a heavy chain amino acid sequence at least 95% identical to SEQ ID NO. 4 and a light chain amino acid sequence at least 95% identical to SEQ ID NO. 5, a heavy chain amino acid sequence at least 95% identical to SEQ ID NO. 6 and a light chain amino acid sequence at least 95% identical to SEQ ID NO. 7, a heavy chain amino acid sequence at least 95% identical to SEQ ID NO. 8 and a light chain amino acid sequence at least 95% identical to SEQ ID NO. 9, a heavy chain amino acid sequence at least 95% identical to SEQ ID NO. 10 and a light chain amino acid sequence at least 95% identical to SEQ ID NO. 11, a heavy chain amino acid sequence at least 95% identical to SEQ ID NO. 12 and a light chain amino acid sequence at least 95% identical to SEQ ID NO.13, and combinations thereof.

15. The method of claim 13, wherein the at least one isolated monoclonal antibody is chosen from the group of antibodies having a heavy chain amino acid sequence at least 95% identical to SEQ ID NO. 4 and a light chain amino acid sequence at least 95% identical to SEQ ID NO. 5, a heavy chain amino acid sequence at least 95% identical to SEQ ID NO. 6 and a light chain amino acid sequence at least 95% identical to SEQ ID NO. 7, a heavy chain amino acid sequence at least 95% identical to SEQ ID NO. 8 and a light chain amino acid sequence at least 95% identical to SEQ ID NO. 9, a heavy chain amino acid sequence at least 95% identical to SEQ ID NO. 10 and a light chain amino acid sequence at least 95% identical to SEQ ID NO. 11, a heavy chain amino acid sequence at least 95% identical to SEQ ID NO. 12 and a light chain amino acid sequence at least 95% identical to SEQ ID NO.13, and combinations thereof.

16. The method of claim 9, wherein the composition further comprises an anti-cancer therapeutic.


SEQUENCE LISTING (23 sequences)

SEQ ID NO: 1 - protein, 279 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 3 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 122 aa, Homo sapiens
EVQLVESGGDLVKPGGSLKLSCAASGFTFSNYGMSWVRQTPDKRLEYVATINSGGSYTYYPDSVKGRFTISRDNAKTTLYLQMSSLKSEDTAMYYCARIYDYDGDYYALDYWGQGTSVTVSS

SEQ ID NO: 5 - protein, 112 aa, Homo sapiens
DVLMTQTPLSLPVSLGDQASISCRSSQSIVHTNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 6 - protein, 122 aa, Homo sapiens
EVQLVESGGALVKPGGSLKLSCTASGFTFTTYAMSWVRQTPERRLEWVATISTYGTYTYYPDSVKGRFTISRDNAKNTLYLQMSSLRSEDTAMYYCARRTMITATDNYFDYWGQGTTLTVSS

SEQ ID NO: 7 - protein, 113 aa, Homo sapiens
DIVLTQSPSSLTVTAGEKVTMSCKSSQSLLNSGNQKNYLTWYQQKLGQSPKLLIYWASTRESGVPDRFTGSGSGTDFTLTISSVQAEDLAVYYCQNDYDYPLTFGAGTKLELK

SEQ ID NO: 8 - protein, 116 aa, Homo sapiens
QIQLVQSGPDLKKPGETVKISCKASGYTFTAYSIHWVKQAPGKGLKWMGWINTETGEATYADDFKGRFALSLATSASTAYLQIKNLKNEDTATYFCNRGVVVPGYWGQGTTLTVSS

SEQ ID NO: 9 - protein, 112 aa, Homo sapiens
DVLMTQTPLSLPVSLGDQASISCKSSQSIVLGNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 10 - protein, 117 aa, Homo sapiens
QIQLVQSGPELKKPGETVKISCKASGYTFTDYSMHWVKQAPGKGLKWMGLINTETGEPTYADDFKGRFAFSLDASASTAYLQINNLENEDTATYFCARTFTPVGQPWGQGTLVTVSA

SEQ ID NO: 11 - protein, 113 aa, Homo sapiens
DIVLTQSPSSLTVTAGERVTMNCKSSQSLLNRGNQRNYLTWYQQKPGQPPKLLIYWASTRESGVPDRFTGSGSGTDFTLTISSVQAEDLAVYYCQNDYSYPLTFGTGTKLELK

SEQ ID NO: 12 - protein, 122 aa, Homo sapiens
EVQLVESGGGLVNPGGSLKLSCAASGFTFTNYAMSWVRQTPEKRLEWVATISSGGSYTYYPDSVKGRFTISRDNAKNTLYLQMSSLRSEDTAMYYCARRTMITATDNYFDYWGQGTILTVSS

SEQ ID NO: 13 - protein, 107 aa, Homo sapiens
DIQMTQSPASLSASVGETVTITCRASENIYSYLAWYQQKQGKSPQLLVYNAKTLAEGVPSRFTGSGSGTDFTLKISRVEAEDLGVYYCVQGTHFPLTFGAGTKLEVK

SEQ ID NO: 14 - DNA, 366 bp, Homo sapiens
gaggtgcagctggtggagtctgggggagacttagtgaagcctggagggtccctgaaactctcctgtgcagcctctggattcactttcagtaactatggcatgtcttgggttcgccagactccagacaagaggctggagtatgtcgcaaccattaatagtggtggtagttacacctactatccagacagtgtgaaggggcgattcaccatctccagagacaatgccaagaccaccctgtacctgcaaatgagcagtctgaagtctgaggacacagccatgtattactgtgcaagaatctatgattacgacggggattactatgctttggactactggggtcaaggaacctcagtcaccgtctcctca

SEQ ID NO: 15 - DNA, 336 bp, Homo sapiens
gatgttttgatgacccaaactccactctccctgcctgtcagtcttggagatcaagcctccatctcttgcagatctagtcagagcattgtacatactaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 16 - DNA, 366 bp, Homo sapiens
gaagtgcagttggtggagtctgggggagccttagtgaagcctggagggtccctgaaactctcctgtacagcctctggattcactttcactacctatgccatgtcttgggttcgccagactccggagaggaggctggagtgggtcgcaaccattagtacttatggtacttatacctactatccagacagtgtgaagggtcgattcaccatctccagagacaatgccaagaacaccctgtacctgcaaatgagcagtctgaggtctgaggacacggccatgtattactgtgcaagacggactatgattacggcgacggataactactttgactactggggccaaggcaccactctcacagtctcctca

SEQ ID NO: 17 - DNA, 339 bp, Homo sapiens
gacattgtgctgacacagtctccatcctccctgactgtgacagcaggagagaaggtcactatgagctgcaagtccagtcagagtctgttaaacagtggaaatcaaaagaattacttgacctggtatcagcagaaactagggcagtctcctaaactgttgatctactgggcatccactagggaatctggagtccctgatcgcttcacaggcagtggatctggaacagatttcactctcaccatcagcagtgtgcaggctgaagacctggcagtttattactgtcagaatgattatgattatccgctcacgttcggtgctgggaccaagctggagttgaaa

SEQ ID NO: 18 - DNA, 348 bp, Homo sapiens
cagatccagttggtgcagtctggacctgacctgaagaagcccggagagacagtcaagatctcctgcaaggcttctggttataccttcacagcctattcaatacactgggtgaagcaggctccaggaaagggtttaaagtggatgggctggataaacactgagacgggtgaggcaacatatgcagatgacttcaagggacggtttgccctgtctttggcaacctctgccagcactgcctatttacagatcaagaacctcaaaaatgaagacacggctacatatttctgtaatagaggggtagtagttcctgggtactggggccaaggcaccactctcacagtctcctca

SEQ ID NO: 19 - DNA, 336 bp, Homo sapiens
gatgttttgatgacccaaactccgctctccctgcctgtcagtcttggagatcaagcctccatctcttgcaaatctagtcagagtattgtacttggtaatggaaacacctatttagaatggtacctacagaagccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 20 - DNA, 351 bp, Homo sapiens
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggcttgataaacactgagactggtgaaccaacatatgcagatgacttcaagggacggtttgccttctctttggacgcctcagccagcactgcctatttgcagatcaacaacctcgaaaatgaggacacggctacatatttctgtgctagaacctttactccggtagggcagccctggggccaagggactctggtcactgtctctgca

SEQ ID NO: 21 - DNA, 339 bp, Homo sapiens
gacattgtgttgacacagtctccatcctccctgactgtgacagcaggagagagggtcactatgaattgcaagtccagtcagagtctgttaaacaggggaaatcaaaggaattacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtactgggaccaagctggagctgaaa

SEQ ID NO: 22 - DNA, 366 bp, Homo sapiens
gaagtgcagctggtggagtctgggggaggcttagtgaaccctggagggtccctgaaactctcctgtgcagcctctggattcactttcactaactatgccatgtcttgggttcgccagactccggagaagaggctggagtgggtcgcaaccattagtagtggtggtagttacacctactatccagacagtgtgaagggtcgattcaccatctccagagacaatgccaagaacaccctgtacctgcaaatgagcagtctgaggtctgaggacacggccatgtattactgtgcaagacggactatgattacggcgacggataactactttgactactggggccaaggcaccattctcacagtctcctca

SEQ ID NO: 23 - DNA, 321 bp, Homo sapiens
gacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcactggcagtggatcaggaacagattttacactgaaaatcagcagagtggaggctgaggatttgggagtttattactgcgtgcaaggtacacattttccgctcacgttcggtgctgggaccaagctggaggtgaaa